(19) United States
(12) Patent Application Publication
(10) Pub. No.: US 2026/0034205 A1
(43) Pub. Date: Feb. 5, 2026

(54) MODIFIED LIVE ATTENUATED FLU VIRUS VACCINES AND USES THEREOF

(71) Applicants: The United States of America, as represented by the Secretary of Agriculture, Washington, DC (US); UNIVERSITY OF GEORGIA RESEARCH FOUNDATION, INC., ATHENS, GA (US)

(72) Inventors: DARRELL R KAPCZYNSKI, WATKINSVILLE, GA (US); DANIEL R PEREZ, ATHENS, GA (US); C. JOAQUIN IGNACIO CACERES SALGADO, ATHENS, GA (US); DANIELA DE SOUZA RAJAO, ATHENS, GA (US); NANY STIVALIS CARDENAS GARCIA, ATHENS, GA (US)

(21) Appl. No.: 19/281,807

(22) Filed: Jul. 28, 2025

Related U.S. Application Data
(60) Provisional application No. 63/677,038, filed on Jul. 30, 2024.

(51) Int. Cl. A61K 39/145; A61K 39/00; A61P 31/16; C07K 14/005; C07K 14/47; C07K 14/54; C12N 7/00

(52) U.S. Cl. CPC A61K 39/145; A61P 31/16; C07K 14/005; C07K 14/4705; C07K 14/54; C12N 7/00; A61K 2039/5256; A61K 2039/575; C07K 2319/21; C12N 2760/16121; C12N 2760/16122; C12N 2760/16134

(57) ABSTRACT

Disclosed herein are modified live virus (MLV) avian influenza reassortment-impaired, non-transmissible, immunogenic, and protective vaccines comprising the modification or rearrangement of internal influenza virus gene segments, incorporation of the DRPAVIAN peptide marker into HA, or modification of NA to encode the chicken interleukin-18 gene. Disclosed are also methods of preparing such MLVs and methods of using such MLVs for the treatment or prevention of influenza in a vertebrate.

### CROSS-REFERENCES TO RELATED APPLICATIONS

This application claims priority benefit from U.S. Provisional Patent Application No. 63/677,038 filed on Jul. 30, 2024. The contents of this patent application are hereby expressly incorporated by reference in their entirety.

### STATEMENT OF FEDERALLY FUNDED RESEARCH

The work described herein was performed with U.S. Government support from the National institute of Food and Agriculture (NIFA) grant No. AFRI-06772. The government has certain rights in this work.

### TECHNICAL FIELD

The disclosure relates modified live attenuated Flu virus vaccines to protect poultry against influenza viruses, kits comprising such modified live attenuated Flu viruses vaccines, and methods of using such modified live attenuated Flu virus vaccines to protect poultry against influenza viruses.

### SEQUENCE LISTING

The instant application contains a Sequence Listing XML required by 37 C.F.R. § 1.831(a) which has been submitted in XML file format via the USPTO patent electronic filing system, and is hereby incorporated by reference in its entirety. The XML file was created on Jul. 30, 2024, is named 0086_23_Sequence_Listing, and has 13,000 bytes.

### BACKGROUND OF THE INVENTION

Influenza A (AI) viruses are genetically diverse pathogens that can infect various hosts including birds, swine, and humans. Vaccines and vaccination have emerged during the past three decades as essential tools in AI control. Their use in poultry can increase resistance to infection, prevent illness and death, reduce virus replication and shed, and reduce virus transmission to birds and mammals, including humans. Historically, only killed influenza virus vaccines are used in animals due to risk of reversion to an enhanced virulent state or recombination with other influenza viruses. In addition, highly pathogenic forms of the virus are not used as vaccine due to concerns over manufacturing issues with regards to potential release from improper inactivation. Recently, recombinant (also known as vectored) influenza virus vaccines have been licensed for use in poultry.

These vaccines express a single key influenza immunogen, known as the hemagglutinin (HA) protein. This protein is responsible for attachment of the virus to host cells, thus immunity against this protein induces protection from disease. Killed (inactivated) influenza virus vaccines only induce systemic antibodies against the virus, whereas a live replicating vaccine virus induces both antibodies and cellular immunity. However, recombinant vaccines only induce immunity against the HA protein.

With regards to poultry, from 2002-2010, over 113 billion doses of avian influenza vaccines were applied to poultry. The majority of these vaccines were used in China, and the main type of the vaccines were killed influenza virus vaccines. In addition, in the U.S, killed influenza virus vaccines are applied in the turkey sector, but not yet in the chicken sector. Thousands of doses are applied in the U.S. yearly. In addition, the swine industry also vaccinates against influenza virus, and vaccinations are routinely carried out in the U.S.These vaccines express a single key influenza immunogen, known as the hemagglutinin (HA) protein. This protein is responsible for attachment of the virus to host cells, thus immunity against this protein induces protection from disease. Killed (inactivated) influenza virus vaccines only induce systemic antibodies against the virus, whereas a live replicating vaccine virus induces both antibodies and cellular immunity. However, recombinant vaccines only induce immunity against the HA protein.

Outbreaks of highly pathogenic avian influenza (HPAI) virus have increased globally beginning in 2002. The virus has become more adapted to its natural host, wild migrating birds, which has resulted in increased spread of the virus to commercial poultry. For example, more than 150 million poultry were killed in Southeast Asia during 2003-2004 HPAI H5N1 outbreaks with losses estimated at US $10 billion. Thailand suffered the largest economic losses recording total estimates of about US $1.2 billion. Trade losses were severe in Thailand, which accounted for a significant amount of loses since the country had been the world's fifth largest poultry exporter in the world. In Korea, the economic losses associated with HPAI outbreaks were largest in 2016-2017 with expected estimates of at least $435 million and maximum of $1.3 billion.

In the U.S. from 2014-2015, an outbreak of HPAI resulted in the death of over 58 million birds, the majority being commercial poultry, resulting in an economic loss of approximately US $3.3 billion to the industry that included US $1.6 billion from destroyed turkeys and eggs. Vaccines were not applied during this outbreak due to trade issues, but were developed and considered.

Thus, new influenza virus modified live virus vaccines that induce mucosal immunity, systemic antibodies, and cellular immunity against influenza are urgently needed.

### SUMMARY OF THE INVENTION

Provided herein are modified live attenuated Flu virus vaccines to protect poultry against influenza viruses, kits comprising such modified live attenuated Flu viruses vaccines, and methods of using such modified live attenuated Flu virus vaccines to protect poultry against influenza viruses.

In an embodiment, the disclosure relates to recombinant influenza virus comprising a modified gene segment 2 with at least one of a polynucleotide encoding a histidine tag or a polynucleotide encoding M42 at the C-terminus of the PB1 open reading frame (ORF); or a modified gene segment 4 containing an IgA-inducing protein (IGIP) coding sequence; or a modified gene segment 6 containing a chicken IL-18 coding sequence. In some embodiments of the disclosure, the modified gene segment 2 in the recombinant influenza virus further comprises at least one of K391E, D581G, and A661T temperature sensitive mutations.

In an embodiment, the disclosure relates to a method of treating a vertebrate in need thereof to prevent or ameliorate one or more symptoms of influenza virus infection, the method comprising administering to the subject a recombinant influenza virus described herein. In some embodiments of the disclosure, the vertebrate in need thereof to prevent or ameliorate one or more symptoms of influenza virus infection are birds, Canidae, Cetacea, Felidae, Mustelidae, Rodentia, Equidae, Bovidae, Suidae, or Primates. In some embodiments of the disclosure, the birds in need thereof to prevent or ameliorate one or more symptoms of influenza virus infection are water fowl, chickens, or turkeys. In some embodiments of the disclosure, the vertebrates in need thereof to prevent or ameliorate one or more symptoms of influenza virus infection are mammals. In some embodiments of the disclosure, the mammals in need thereof to prevent or ameliorate one or more symptoms of influenza virus infection are pigs, horses, whales, dolphins, or humans.

### BRIEF DESCRIPTION OF THE DRAWINGS

These and other features, aspects, and advantages of the present disclosure will become better understood with regard to the following description, appended claims, and accompanying drawings.

FIG. 1 depicts a diagrammatic summary of the influenza Segment 7 transcript and Open Reading Frame (ORF) splice variants. The nucleotide coordinates of splice sites are shown. Potential ORFs are shown in gray, and the total amino acid sizes are given to the right.

FIG. 2A and FIG. 2B depict schematic diagrams of the modified viral segments in attΔM2 and the RAMΔM2. FIG. 2A shows diagrams of the modified segments in attΔM2, with segment 2 shown at the top, modified segment 1 shown at the center, and segment 7 shown at the bottom. The attΔM2 carries temperature-sensitive mutations (ts) in PB1 (segment 2; K391E, D581G, and A661T) and PB2 (segment 1; N265S), an HA tag at the C-terminus of PB1, and a modification of the M segment (segment 7) that precludes M2 synthesis. FIG. 2B shows diagrams of the modified segments in RAMΔM2, with rearranged segment 2 shown at the top, and rearranged segment 7 shown at the bottom. Proteolytic cleavage leads to excision of M42 from PB1. A series of stop codons prevents synthesis of M2/M42 from segment 7.

FIG. 3 depicts a diagrammatic summary of the HA and NA segments engineered to contain molecular signatures on their respective major ORFs and additional modifications to accommodate foreign genes. In this case, coding sequences for the IgA-inducing protein (IGIP) and chicken IL-18 are incorporated into the HA and NA segments, respectively.

FIG. 4 depicts an image of the RT-PCR fragment profiles of MLVs before serial passages in eggs (E1) or after serial passages in eggs (E5) targeting the PB1-M2 (top), HA-DRPAVIAN (middle), and NA-IL-18 (bottom). Wild-type segments were included as positive controls to demonstrate the differences in size between modified and non-modified segments. Note that band sizes are similar in E1 and E5 for all segments and vaccine candidates analyzed.

FIG. 5A and FIG. 5B depict graphs of the growth kinetics profiles of MLVs in MDCK cells titrated by RT-qPCR. FIG. 5A shows data obtained at 37° C. FIG. 5B shows data obtained at 41° C. Filled triangles (▴) denote MLV-H9N2-IL data; filled squares (▪) denote MLV-H9N2; empty diamonds (⋄) denote WF10 att; and filled circles (●) denote WF10 wt. The Y axis shows the mean Log 10 TCID₅₀ equivalent/mL. The X axis shows the time post infection in hours (h). Samples with undetected virus titers were assigned the limit of detection value (0.699 Log 10 TCID₅₀ equivalent/mL).

FIG. 6 depicts an image of the IL-18 expression from the MLV-H9N2-IL virus by western blot. MDCK cells were inoculated as follows: lane 1 (MLV-H9N2-IL; MOI 1), lane 2 (MLV-H9N2-IL; MOI 10), lane 3 (MLV-H9N2; MOI 1), lane 4 (MLV-H9N2; MOI 10). Lane 5, protein lysates of HEK293T cells transfected with the control expression plasmid pCAGGS expressing N2WF10-Furin-2A-IL-18. Lane 6, negative control cell lysates. The arrows indicate the predicted molecular weight of chicken IL-18 (23 KDa) and the host cellular protein glyceraldehyde-3-phosphate dehydrogenase (GAPDH; 37 KDa), which is shown as a gel loading control. MWM is the molecular weight marker.

FIG. 7A to FIG. 7D depicts graphs of the reassortment data upon co-infection of MDCK cells or 2-week-old chickens with MLV-H9N2 or MLV-H9N2-IL and H9N2 WT virus. FIG. 7A shows the proportion of NGS reads that matched MLV or WT for each specific segment upon co-infection of MDCK cells with MLV-H9N2 and H9N2 WT virus. Filled squares (▪) denote MLV-H9N2; empty circles (∘) denote WT. FIG. 7B shows the proportion of NGS reads that matched MLV or WT for each specific segment upon co-infection of MDCK cells with MLV-H9N2-IL and H9N2 WT virus. Filled triangles (▴) denote MLV-H9N2-IL; empty circles (∘) denote WT. FIG. 7C shows the proportion of NGS reads that matched MLV or WT for each specific segment upon co-infection of 2-week-old chickens with MLV-H9N2 and H9N2 WT virus. Filled squares (▪) denote MLV-H9N2; empty circles (∘) denote WT. FIG. 7D shows the proportion of NGS reads that matched MLV or WT for each specific segment upon co-infection of MDCK cells with MLV-H9N2-IL and H9N2 WT virus at a 10:1 ratio. Filled triangles ( ) denote MLV-H9N2-IL; empty circles denote H9N2 WT.

FIG. 8A and FIG. 8B depict graphs of the virus transmission between directly inoculated and direct contact animals in oropharyngeal (OP) swabs and cloacal (CL) swabs. FIG. 8A shows data for OP swabs. FIG. 8B shows data for CL swabs. Swabs were collected (n=4/group/day) every day from 1 to 6 days post-inoculation/days post-contact to analyze transmission between directly inoculated and direct contact animals. Filled triangles (▴) denote MLV-H9N2-IL data; filled squares (▪) denote MLV-N9N2 data; empty circles (∘) denote mock data. Viral loads were analyzed by RT-qPCR. Virus titers are shown as the mean±SD Log 10 TCID50 equivalent/mL.

FIG. 9 depicts a graph of the Hemagglutination Inhibition (HI) titer in chickens 14 days post direct inoculation with either the MLV-H9N2, MLV-H9N2-IL, or mock inoculated, and in chickens 14 days post-contact. The Y axis shows the HI titer. The X axis shows the treatments. Filled triangles (▴) denote MLV-H9N2-IL data; filled squares (▪) denote MLV-N9N2 data; empty circles (∘) denote mock data.

FIG. 10 depicts a graph of the virus titer in OP and CL swabs collected from vaccinated chicks three (3) and five (5) days after homologous challenge. Y axis shows the Log 10 TCID50 equivalent/mL. The X axis shows the samples. Bars 1, 5, 9, and 13 correspond to MLV-H9N2-IL-vaccinated animals; bars 2, 6, 10, and 14 correspond to MLV-H9N2-vacinated animals; bars 3, 7, 11, and 15 correspond to WT H9N2-vaccinated animals; and bars 4, 8, 12, and 16 correspond to mock-inoculated animals. Virus titers are shown as the mean±SD Log 10TCID50/mL. Samples with undetected virus titers were assigned the limit of detection value (0.699 Log 10 TCID50/mL). Filled triangles (▴) denote MLV-H9N2-IL data; filled squares (▪) denote MLV-N9N2 data; filled circles (●) denote WT H9N2 data; empty circles (∘) denote mock data.

FIG. 11A to FIG. 11C depict graphs of the HI titer, virus neutralization, and NP ELISA in blood samples taken at 12 dpv and 12 dpb from the WIVadj-H9N2 (crossed empty squares ( [symbol] )), MLV-H9N2 (filled squares (▪)), and MLV-H9N2-IL (filled triangles (▴)), and mock vaccinated (empty circles (∘)). FIG. 11A shows data for hemagglutination inhibition (HI) assays. The Y axis shows the HI titer. The X axis shows the treatment. Bar 1 MLV-H9N2-IL 12 dpv, bar 2 MLV-H9N2 12 dpv, bar 3 WIVadj-H9N2 12 dpv, bar 4 Mock 12 dpv, bar 5 MLV-H9N2-IL 12 dpb, bar 6 MLV-H9N2 12 dpb, bar 7 WIVadj-H9N2 12 dpb, bar 8 Mock 12 dpb.

FIG. 11B shows nanoluciferase activity (VNluc) data as virus neutralization measurement. VNluc titers were plotted as the inhibitory sera dilution 50 (ISD50) and were displayed in the graph. The Y axis shows the Log₁₀ RLU (AU). The X axis shows the Log₂ serum dilution factor. FIG. 11C shows the NP ELISA results. The Y axis presents the S/N ratio. The X axis shows the treatments.

FIG. 12 depicts a graph of the viral loads in the sinuses, trachea, lungs, pancreas, and cloaca samples collected at 3 days post-challenge with 10⁸ EID50/chicken of wild type H9N2 strain. Data for WIVadj-H9N2 is shown with crossed squares ( [symbol] ), data for MLV-H9N2 is shown with filled squares (▪), data for MLV-H9N2-IL is shown with filled triangles (▴), data for mock-vaccinated/challenge with wild type H9N2 (Mock-Challenge) is shown with filled circles (●), and data for mock vaccinated/non-challenge (Mock) is shown with empty circles (∘). The Y axis shows the log₁₀ TCID₅₀/g tissue. Data for MLV-H9N2-IL is shown in bars 1, 6, 11, 16, and 21; data for MLV-H9N2 is shown in bars 2, 7, 12, 17, and 22; data for WIVadj-H9N2 is shown in bars 3, 8, 13, 18, and 23; data for mock challenge is shown in bars 4, 9, 14, 19, and 24; and data for mock is shown in bars 5, 10, 15, 20, and 25. (n=4/group)

FIG. 13A and FIG. 13B depict graphs of the virus titer in OP swabs and CL swabs after challenge with 10⁸ EID50/chicken of wild type H9N2 strain. WIVadj-H9N2 (crossed squares ( [symbol] )), MLV-H9N2 (filled squares (▪)), MLV-H9N2-IL (filled triangles (▴)), mock-vaccinated/challenge with wild type H9N2 (Mock-Challenge, filled circles (●)) and mock vaccinated/non-challenge (Mock, empty circles (∘)) . . . . FIG. 13A shows the virus titer in OP swabs. FIG. 13B shows the virus titer in CL swabs. The Y axis shows the log₁₀ TCID₅₀eq/mL. The X axis shows the treatments. Data analysis and graphs were prepared using Prism v9. Ordinary one-way ANOVA was performed to calculate P values followed by Tukey's multiple comparison test. Significant differences between vaccine groups and the non-vaccinated/challenge group are indicated with p≤0.05 considered significant. (n=6/group).

FIG. 14 depicts a graph of the virus replication in animals primed or primed and boosted with low dose (10⁴ EID₅₀/mL, filled triangles (▴)) or high dose (10⁶ EID₅₀/mL, filled squares (▪)) in OP swabs or CL swabs. The Y axis presents the log₁₀ TCID₅₀/mL. The X axis presents the samples: data for low dose is shown in bars 1, 4, 7, and 10; data for high dose is shown in bars 2, 5, 8, and 11; and data for mock is shown in bars 3, 6, 9, and 12, (n=6/group).

FIG. 15 depicts graphs of virus titers in vaccine samples mixed with distilled water and incubated at various temperatures (25° C., 30° C., 40° C., and 50° C.) for 2 hours with 10⁶ EID₅₀/mL, (filled triangles (▴)) or 10⁴ EID₅₀/mL (empty triangles (Δ)), or mock-vaccinated (empty circles (∘)) . . . . The incubation temperature is indicated above each graph. The Y axis shows the log₁₀ TCID₅₀/mL. The X axis shows the time in hours (h).

FIG. 16 depict a graph of the HI titers in blood samples collected 12 dpv, 26 dpv, and 12 dbp of animals vaccinated via drinking water with low dose of high dose vaccine. Empty symbols correspond to the 10⁴ EID₅₀/mL low MLV-H9N2-IL dose as follows: 12 days post-prime (12 dpp, empty triangles (Δ)), 26 dpp (empty squares (□)), 12 days post-bost (12 dpb, empty polygons ( [symbol] )). Filled symbols correspond to the 10⁶ EID₅₀/mL high MLV-H9N2-IL dose as follows: 12 dpp, (filled triangles (▴)), 26 dpp (filled squares (▪)), 12 days post-bost (12 dpb, filled polygons ( [symbol] )). Mock controls represented with empty circles (∘). (n=6/group)

FIG. 17A and FIG. 17B depict graphs of the virus neutralization titers and NP ELISA results animals primed or primed and boosted with low dose (10⁴ EID₅₀/mL) or high dose (10⁶ EID₅₀/mL) virus in drinking water. FIG. 17A shows the virus neutralization titers. The Y axis shows the Log₁₀ RLU (AU). The X axis shows the Log₂ serum dilution factor. 10⁴ EID₅₀/mL low MLV-H9N2-IL dose 12 dpp (empty triangles (Δ)), 26 dpp (empty squares (□)), 12 dpp (empty polygons ( [symbol] )). 10⁶ EID₅₀/mL high MLV-H9N2-IL 12 dpp, (filled triangles (▴)), 26 dpp (filled squares (▪)), 12 dpb (filled polygons ( [symbol] )). Mock controls represented with empty circles (∘). FIG. 17B shows the NP ELISA results. The Y axis shows the S/N ratio. The X axis shows the treatments: 10⁴ EID₅₀/mL low MLV-H9N2-IL dose (empty triangles (Δ)), 10⁶ EID₅₀/mL high MLV-H9N2-IL dose (filled triangles (▴)), mock vaccinated (empty circles (∘)).

FIG. 18 depict graphs of the viral load in sinuses, trachea, and cloaca of animals primed or primed and boosted with low dose or high dose virus in drinking water. The Y axis shows the log₁₀ TCID50/g tissue. The X axis shows the treatments: 10⁴ EID₅₀/mL low MLV-H9N2-IL dose/challenge (empty triangles (Δ)), 10⁶ EID₅₀/mL high MLV-H9N2-IL dose/challenge (filled triangles (▴)), mock-vaccinated/challenge (filled circles (●)), and mock-vaccinated/non-challenged (empty circles (∘)). Data analysis and graphs were prepared using Prism v9. Significant differences between vaccine groups and the non-vaccinated/challenge group are indicated with p≤0.05 considered significant. (n=4/group).

FIG. 19A and FIG. 19B depict graphs of the virus titers in OP swabs and CL swabs collected (n=4/group/day) every other day from 1 to 5 dpc and titrated by TCID50. as the Y axis shows the mean±SD Log 10 TCID50/mL. The X axis shows the treatments: 10⁴ EID₅₀/mL low MLV-H9N2-IL dose/challenge (empty triangles ( )), 10⁶ EID₅₀/mL high MLV-H9N2-IL dose/challenge (filled triangles ( )), mock-vaccinated/challenge (filled circles ( )), and mock-vaccinated/non-challenged (empty circle ( )). Samples with undetected virus titers were assigned the limit of detection value (0.699 Log 10 TCID50/mL). Data analysis and graphs were prepared using Prism v9. Ordinary one-way ANOVA was performed to calculate P values followed by Tukey's multiple comparison test. Significant differences between vaccine groups and the non-vaccinated/challenge group are indicated with p<0.05 considered significant. (n=4/group).

### BRIEF DESCRIPTION OF THE SEQUENCE LISTING

The nucleotide sequences disclosed in the specification are listed in Table 1, below.

| Sequence Identifier | Type | Description |
|---|---|---|
| SEQ ID NO: 1 | amino acid | G4S |
| SEQ ID NO: 2 | amino acid | DRPAVIAN |
| SEQ ID NO: 3 | DNA | primer PB1-2068F |
| SEQ ID NO: 4 | DNA | primer Bm-PB1-2341R-eq |
| SEQ ID NO: 5 | DNA | primer H9HA-1F |
| SEQ ID NO: 6 | DNA | primer H9HA657-693Rev |
| SEQ ID NO: 7 | DNA | primer WF10NA118F |
| SEQ ID NO: 8 | DNA | primer N2Hu_1463R |
| SEQ ID NO: 9 | DNA | primer M + 25 |
| SEQ ID NO: 10 | DNA | primer M − 124 |
| SEQ ID NO: 11 | DNA | probe M + 64 |
| SEQ ID NO: 12 | DNA | Primer Opti1-F1 |
| SEQ ID NO: 13 | DNA | Primer Opti1-F2 |
| SEQ ID NO: 14 | DNA | Primer Opti1-R1 |

### DETAILED DESCRIPTION

Disclosed herein are recombinant influenza A virus comprising a chimeric gene segment 2 encoding the PB1 open reading frame (ORF) with a self-cleavable C-terminal end peptide followed by either the M2 or the M42 ORF, and a gene segment 7 expressing only the M1 protein, and use of such recombinant influenza A viruses.

The Orthomyxoviruses are a family of RNA viruses that includes seven genera: Influenza virus A, Influenza virus B, Influenza virus C, Influenza virus D, Isavirus, Thogotovirus, and Quaranjavirus. Influenza A virus is one of the world's major uncontrolled pathogens, causing seasonal epidemics as well as global pandemics. Influenza A viruses can infect various vertebrate hosts including birds and mammals. Influenza vertebrate hosts are birds, Canidae, Cetacea, Felidae, Mustelidae, Rodentia, Equidae, Bovidae, Suidae, and Primates. In some embodiments the vertebrates are birds such as water fowl, chickens, or turkeys. In some embodiments the vertebrates are mammals such as pigs, horses, whales, dolphins, or humans. In some embodiments the vertebrates are humans. In some embodiments the vertebrates are chickens, turkeys, dolphins, whales, swine, horses, or humans.

The genome of the Influenza A virus is negative-sense, single-stranded, segmented RNA. The Influenza A subtypes are named (HxNy) according to the type of hemagglutinin (H) and the type of neuraminidase (N) present in the virus. Up to date 16 different H antigens and 9 different N antigens are known.

The Influenza virus particle (also called a virion) is made of a viral envelope wrapped around a central core. The outer layer of the influenza virion is a lipid membrane taken from the host cell in which the virus multiplies. Inserted into the lipid membrane are the hemagglutinin (HA) protein, the neuraminidase (NA) protein, and the matrix-2 (M2) protein. Four M2 proteins form a proton-selective ion channel where the units are helices stabilized by two disulfide bonds. Beneath the lipid membrane is a layer of the matrix protein (M1) forming a shell. Within the interior of the virion are eight negative strand viral RNA segments consisting of RNA joined with the nucleoprotein (NP), and the three polymerase subunits (Polymerase Basic protein1 (PB1), Polymerase Basic protein 2 (PB2), and polymerase acidic protein (PA)). Non-Structural protein 1 (NS1) and Non-Structural protein 2 (NS2) are found inside the virion.

The influenza A virus gene segment 1, also referred to as PB2 gene segment, encodes the cap-binding transcriptase PB2. Using alternative translation initiation sites, gene segment 2, also referred to as PB1 gene segment, encodes elongation-associated proteins PB1, PB1-F2, and PB1-N40. By a ribosomal frameshift gene segment 3, also referred to as PA gene segment, encodes the polymerase acidic proteins PA and PA-X, and by using alternative translation sites and N-terminal truncation encodes PA-N155 and PA-N182. Gene segment 4, also referred to as HA gene segment, encodes the hemagglutinin protein HA. Gene segment 5, also referred to as NP gene segment, encodes the RNA binding nucleoprotein NP. Gene segment 6, also referred to as NA gene segment, encodes the neuraminidase protein NA. Gene segment 8, also referred to as NS1/NS2 gene, encodes the non-structural protein NS1, and by alternative RNA splicing it encodes NS3 and the nuclear export protein NS2/NEP.

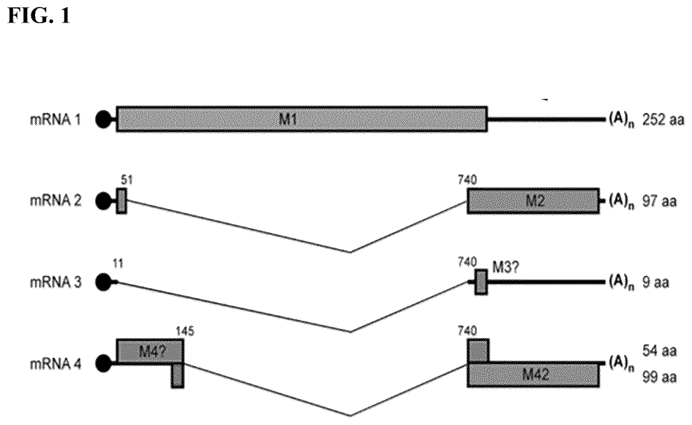

The Influenza A virus genome segment 7 is also referred to as M gene or M1/M2 gene, and produces at least four mRNA transcripts (mRNA1, mRNA2, mRNA3, and mRNA4). A schematic diagram of the influenza segment 7 and its transcripts is shown in FIG. 1. The full-length M transcript is 1,004 nt. The M1 protein is encoded by mRNA1, the colinear transcript from the initiation codon at nucleotides 26 to28 to the termination codon at nucleotides 782 to 784. The M2 protein is encoded by mRNA2 which comprises nucleotides 26 to28 until nucleotide 51, and nucleotides 740 to nucleotide 1007. mRNA3 is interrupted from nucleotides 11 to 740 and is expected to produce a 9-amino acid polypeptide. mRNA4 codes for a third protein (M42), using an initiation codon at nucleotides 114 to 116. Strain-specific single nucleotide changes in the 5′ single strand of segment 7 are said to result in marked alterations of splice site usage. M42 is said to result from leaky ribosomal scanning and to have an antigenically distinct ectodomain that can functionally complement M2 in vitro and in vivo. (Wise et al., 2012, “Identification of a Novel Splice Variant Form of the Influenza A Virus M2 Ion Channel with an Antigenically Distinct Ectodomain,” PLOS Pathogens 8(11): e1002998).

U.S. Pat. No. 11,028,408 discloses a rearranged influenza A viral genome segment 2. The rearranged segment 2 comprises: a) a first influenza type A viral genome segment 2 which comprises a nucleic acid sequence that encodes PB1 and b) a portion of a second influenza type A viral genome segment, wherein the portion of the second influenza type A viral genome segment comprises the portion of influenza type A viral genome segment 8 comprising a nucleic acid sequence that encodes the influenza type A viral protein, NS2, wherein the NS2 nucleic acid sequence is removed from RNA segment 8 of the genome, wherein the NS2 nucleic acid sequence is downstream of the nucleic acid sequence that encodes PB1, wherein a cleavage site is present between the nucleic acid sequence that encodes PB1 and the NS2 nucleic acid sequence, wherein the cleavage site is a 2A-like-cis-acting hydrolase element (CHYSEL) site, and wherein the nucleic acid sequence that encodes PB1 and the NS2 nucleic acid sequence are co-translatable.

U.S. Pat. No. 11,214,799 discloses live attenuated influenza vaccines comprising mutant recombinant influenza virus gene segment 7 with single mutations that modulate the expression of M2 and/or M42. These vaccines protect inoculated birds against viruses from a different lineage, that contain an HA protein with at least 70% similarity.

Live attenuated (att) human influenza viruses for type A and B influenza viruses with cold adapted/temperature sensitive (ts) mutations were developed during the 1960's by serial passage at sequentially lower temperatures in specific pathogen free primary chicken kidney cells. The att influenza viruses that grow efficiently in 10-day-old embryonated chicken eggs at 25° C. were obtained using the strains A/Ann Arbor/6/60 (H2N2) and B/Ann Arbor/1/66. Mutations in PB1, PB2, NP, M, and NS contributed to the att phenotype. More recently, the inventors developed a remarkably stable and efficacious alternative MLV strategy for influenza type A and B viruses (IAV att and IBV att, respectively) with temperature sensitive mutations in PB2 and PB1 and an epitope tag in PB1 [ts+tag=att]. The IAV att strategy shares some ts mutations in common with the temperature sensitive A/Ann Arbor strain but the modifications of the IBV att are unique to the IAV att viruses, not shared with any other temperature sensitive IBV strain. The att strategy has been extensively tested in swine and poultry (among other animal models) with optimal results against aggressive virus challenge. See, for example, U.S. Pat. No. 8,475,807, US Patent Application Publication 2016/0022807, U.S. Pat. No. 10,080,794.

Of particular relevance are the results obtained in chickens and disclosed in Song, H., et al., 2007, “A new generation of modified live-attenuated avian influenza viruses using a two-strategy combination as potential vaccine candidates,” J. Virol. 81: 9238-9248; and Cai, Y. et al., 2011, “Improved hatchability and efficient protection after in ovo vaccination with live-attenuated H7N2 and H9N2 avian influenza viruses,” Virol. J. 8: 31, doi:10.1186/1743-422X-8-31. Highlights are: 1) a single-dose immunization in ovo with the att H5N1 vaccine virus in 18-day-old chicken embryos resulted in more than 60% protection for 4-week-old chickens and 100% protection for 9-to 12-week-old chickens. Boosting at 2 weeks post-hatching provided 100% protection against challenge with the HPAI H5N1 virus for chickens as young as 4 weeks old, with undetectable virus shedding post-challenge; 2) modification of the HA cleavage site in H7 and H9 subtypes in the context of an att backbone led to improved hatchability after a single dose in ovo vaccination of 18-day old eggs, which ultimately provided complete protection against homologous challenge with LPAIV strains at 2 or 6 weeks post-hatching. Taken together, these studies highlight the potential of the att strategy to protect chickens against LPAIV and HPAIV strains.

H5N1 and H9N2 avian influenza subtypes are among the top in the list of the World Health Organization's (WHO) agents with the greatest pandemic potential. Both virus subtypes are entrenched in poultry in Egypt and in other parts of the world where they remain endemic. Inactivated H5N1 vaccines or recombinant H5 vaccines induce inadequate immune responses and limited protection. Live attenuated H5N1 influenza vaccines are promising; however, safety concerns regarding the possibility of reassortment between the H5 HA gene segment with segments from other circulating influenza viruses has prevented further consideration. In order to overcome this major drawback, the authors have designed a highly innovative strategy in order to rearrange the genome of an avian H9N2 influenza virus to allow insertion and expression of foreign genes. This strategy was used to generate a dual H9-H5 influenza-vectored virus (Pena, L. et al., 2013, “Influenza viruses with rearranged genomes as live-attenuated vaccines” J. Virol. 87: 5118-5127, doi:10.1128/JVI.02490-12). The genomic rearrangement was achieved by expressing the NEP/NS2 gene from a single open reading frame (ORF) downstream of the PB1 gene, and the transgene was cloned downstream of a truncated form of the NS1 protein.

Avian influenza viruses (H9N2) expressing reporter genes and the entire ORF of the H5 hemagglutinin were generated by reverse genetics. In vitro studies demonstrated that these vectors had impaired polymerase activity and reduced viral replication. Further in vivo characterization showed that this strategy resulted in virus attenuation. More importantly, immunization with the dual H9-H5 influenza virus resulted in protection against lethal H5N1 challenge in both mice and ferrets. Taken together, these results demonstrate that rearranging the influenza genome has great potential for the development of improved vaccines against influenza as well as other pathogens. A key aspect of the studies with influenza viruses with re-arranged genomes is the over-attenuated nature of the NEP/NS2 re-arrangement which affects in vitro growth and would likely limit vaccine efficacy in poultry. However, the authors have designed a novel MLV-AI strategy, called re-arranged M or RAM, with optimal in vitro growth characteristics and whose vaccine efficacy in poultry is being tested.

The M2 transmembrane protein has been shown to play several key roles in virus replication. It acts as an ion channel that allows for virion acidification for uncoating and mediates virus assembly, budding and release. A variant of M2, identified as M42, was recently identified with an altered ectodomain that can functionally replace M2. M42 supports high titer virus replication in cultured cells and virulence in mice but displays functional differences from M2; in particular an altered subcellular localization that is heavily biased towards the Golgi compartment. M42 is made by leaky ribosomal scanning from an alternatively spliced form of segment 7 mRNA. Production of this splice variant is a rare feature of influenza A viruses but is disproportionally represented in HPAI viruses of various lineages. M2 and M42 expression is balanced by competing use of the mRNA2 and mRNA4 splice donor (SD) sequences and the balance can readily be tipped by single nucleotide polymorphisms in these regions. The ectodomain sequence of M2 is highly conserved amongst all IAV strains, likely reflecting functional constraints, both for itself, the portion of sequence it shares with the M1 polypeptide and overlapping cis-acting RNA signals. Functions attributed to the M2 ectodomain include directing incorporation of the polypeptide into the secretory pathway (in class I topology despite the protein lacking a cleavable signal sequence) as well as into virus particles. Consistent with the conclusion that M2 ectodomain sequences affect interactions with the secretory pathway, as noted above, M42 shows a distinctly different intracellular localization pattern to M2, being noticeably biased towards Golgi residence. The author's previous research utilizing reverse genetics to knock-out M2 expression by G52C substitution demonstrated that AIV could rely solely on M42 for virus propagation. In addition, when tested as a live virus vaccine, both homologous and heterologous protection was observed. See U.S. Pat. No. 11,214,799. Finally, in transmission studies, the M42 vaccine virus was demonstrated to infect and replicate, but not shed to susceptible cohort. These attributes make its selection promising for further candidate analysis.

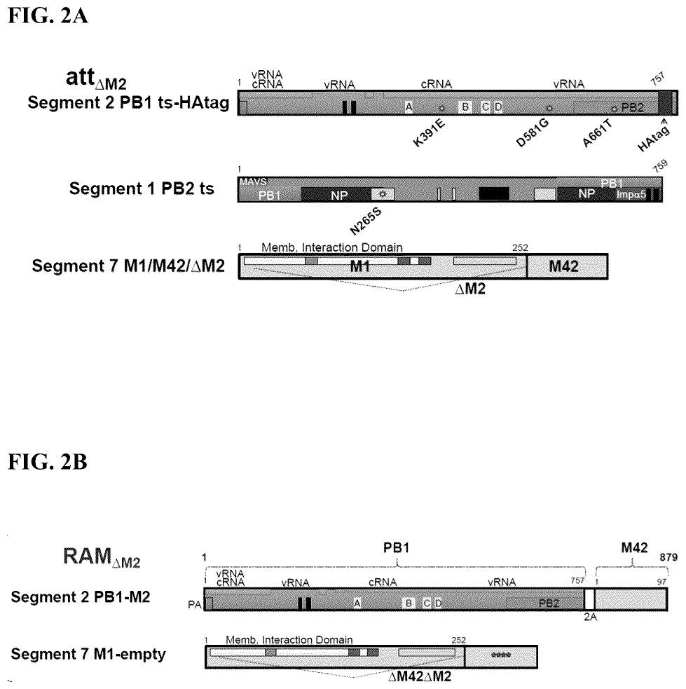

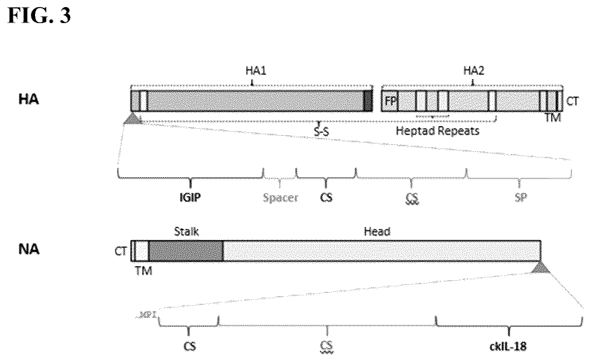

Taking into consideration the concerns of stakeholders and regulatory agencies, the authors have established a set of parameters for an ideal MLV-AI vaccine to: 1) preserve the in vitro growth characteristics of the MLV vaccine similar to wild type levels, 2) maximize mucosal immune responses, 3) incorporate unique molecular diagnostic markers, 4) increase the breath of the immune response, and 5) prevent transmission and reassortment. The authors propose to design these features and compare two different MLV-AI vaccine strategies as described in FIG. 2A and FIG. 2B. In the first strategy, the well characterized att backbone will be produced to contain ts mutations in PB2 and PB1, an HA tag at the C-terminus of PB1, and mutations in segment 7 to eliminate the M2 ORF (attΔM2).

In the second MLV-AI strategy, virus attenuation is achieved through genome rearrangement of segments 2 and 7. In the original approach to this rearranged M segment strategy (called RAM), segment 2 was modified to encode a chimeric PB1-M2 ORF and a segment 7 with multiple stop codons that prevent synthesis of M2/M42. The PB1-M2 chimera is designed in such a way that M2 is post-translationally cleaved from PB1 during virus replication. In addition, the spliced mRNA form that encodes M2 is still produced to preserve the unspliced/spliced mRNA ratios from segment 7 as in the wild-type virus, but the spliced mRNA encodes only a 10 amino acid peptide in common with M1. In the MLV-RAM vaccine, the genetic modifications also prevent the synthesis of the M42 product. MLV-RAM strains with H7N3 and H9N2 surface gene segments grow in 9-day old SPF embryonated chicken eggs to titers similar to their respective wild type strains and are stable over several passages. Segment 2 will be modified to encode a chimeric PB1-M42, whereas segment 7 will remain with multiple early stop codons to prevent the synthesis of M2/M42. This second strategy is called RAMΔM2. The authors hypothesize that the MLV-RAMΔM2 vaccines will grow as efficiently as the original MLV-RAM vaccines (that express M2).

Experimental MLV-AI vaccines are thought to stimulate mucosal and systemic IgA and IgG as well as T-cell mediated immunity because they more closely resemble a natural virus infection, but their relative influence on promoting IgA responses is not well understood. IgA responses are considered of great significance to prevent and/or control a myriad of genital, intestinal, and respiratory infections, including influenza. After a typical influenza infection, both IgA and IgG responses are detected at the airway mucosa with neutralizing activity against influenza. IgA, particularly secretory IgA (sIgA) in its multiple multimeric forms is typically more broadly-neutralizing than IgG. IgA neutralizes pathogens without causing inflammation because of its inability to fix and activate the complement cascade. In contrast, IgG responses that bind but do not neutralize the virus (non-protective antibodies) have been linked to immune complex-mediated disease in middle-aged people that were severely ill after infection with the pandemic influenza or in pigs vaccinated with antigenically-mismatched inactivated vaccines. A better understanding of the role of IgA responses against influenza should help pave the way to universal influenza vaccines. A vaccine strategy aimed at improving mucosal IgA responses against influenza would be ideal to enhance and/or broaden protection against antigenic drifted variants or clades while minimizing the risks associated with suboptimal antibody-antigen match, disease enhancement or immunopathology.

The HA and NA segments may be engineered to contain molecular signatures on their respective major ORFs and additional modifications to accommodate foreign genes. In the instant application, coding sequences for IGIP and chicken IL-18 were incorporated into the HA and NA segments, respectively. The proper expression of the foreign gene is achieved by additional modifications that include spacers, cleavage sites (CS) and signal peptide sequences (SP).

The majority of currently approved and widely used avian influenza vaccination programs worldwide, including those in Mexico, Egypt, Pakistan, and China, rely primarily on adjuvanted inactivated whole virus vaccines. It is well recognized that inactivated vaccines induce robust serum antibody responses but fail to elicit effective mucosal and cell-mediated immunity. Moreover, they are incapable of activating pattern-recognition receptors on infected cells, which play a crucial role in enhancing the production of pro-inflammatory cytokines and type I interferons, thereby initiating the local cellular innate immune response. Another significant drawback is the potential for mismatches between circulating and vaccine strains, allowing the virus to evolve and acquire mutations that could increase its replication and transmission capabilities. Studies have demonstrated that these vaccines fail to reduce virus shedding and do not prevent the transmission of H9N2 in chickens, highlighting their inability to disrupt the influenza transmission cycle.

MLVs hold promise for developing broader and longer-lasting effective vaccines against avian influenza due to their ability to replicate and mimic natural infection. Genome rearrangement is a viable, safe, and efficacious approach for MLV development. In previous studies, our RAM vaccines effectively protected mice against clinical disease and mortality (Cardenas-Garcia S, et al., 2019, “Maternally-Derived Antibodies Protect against Challenge with Highly Pathogenic Avian Influenza Virus of the H7N3 Subtype,” Vaccines (Basel) 7). Additionally, the incorporation of a natural adjuvant into the HA segment enhanced the stimulation of IgG and IgA responses (41).

Building upon these findings, two MLVs were developed and are disclosed herein, MLV-H9N2 and MLV-H9N2-IL, based on genome rearrangement of the H9N2 subtype, the most prevalent LPAIV circulating in chickens worldwide. Utilizing the previously established RAM approach as an attenuated backbone, additional molecular markers were incorporated into the HA to prevent reassortment and an immunomodulator was incorporated into the NA to further stimulate protective antibody responses.

To assess the genetic stability of the MLV-H9N2 and MLV-H9N2-IL viruses, they were subjected to five serial passages in eggs and their genetic integrity was evaluated using RT-PCR and Sanger sequencing. The results confirmed that both viruses maintained their genetic integrity throughout the serial passages.

Interestingly, both the DRPAVIAN modification and the inclusion of IL-18 in the NA resulted in a fitness disadvantage compared to the wild-type (WT) segments from the ck/Tunisia strain in vitro. In contrast, the modified PB1-M2 and M1ΔM2 segments were detected in the progeny virus and exhibited similar behavior to unmodified segments in vitro. Not wishing to be bound by theory, one possible explanation for the presence of these modified MLV segments in the progeny virus is the fact that MLVs were not attenuated in vitro. Despite several gene modifications, both MLVs replicated to similar levels as an H9N2 WT virus at 37° C. and 41° C. in vitro. However, both MLVs exhibited an attenuated profile in vivo, with virus replication levels not exceeding 3 log₁₀ TCID₅₀/mL and restricted to within 3 to 5 days post-vaccination. These low levels of virus replication in directly-inoculated animals prevented the transmission of MLV-H9N2 and MLV-H9N2-IL to naïve direct contact chickens. Consistent with the attenuated profile of MLVs in vivo, the in vivo co-infection data shown here demonstrated that both MLVs have a fitness disadvantage and all MLV segments were outcompeted by the WT virus in vivo. Additionally, no reassortment was observed between the MLV viruses disclosed herein and the WT virus in vivo. Notably, the modified PB1-M2 and M1ΔM2 segments exhibited reduced fitness in vivo, further corroborating their safe profile.

The humoral immune responses elicited by vaccination via the EROC route were also investigated. As anticipated, the WIVadj-H9N2 vaccine induced the highest levels of HI titers and neutralizing antibodies. While both MLVs generated lower HI titers, their responses were consistent with antibody levels considered protective. This aligns with the author's previous studies using alternative MLVs against type A and B influenza viruses, and contrasts with the limited seroconversion typically associated with currently approved cold-adapted seasonal influenza vaccines for human use.

Despite the robust humoral responses in the WIVadj-H9N2 vaccine group, these were not sufficient to fully protect the birds against the homologous H9N2 challenge. Virus was detected in the lungs and OP swabs of birds immunized with the WIVadj-H9N2 vaccine, confirming the inability of inactivated vaccines to reduce viral loads in vaccinated birds. Notably, chickens vaccinated with the WIVadj-H9N2 vaccine exhibited significant virus replication after challenge, in samples collected from the pancreas. This unexpected finding may partially explain the ineffectiveness of inactivated vaccines in the field against H9N2 LPAIVs. Conversely, MLV-vaccinated birds, especially those receiving the MLV-H9N2-IL vaccine, exhibited almost complete protection against the challenge. This highlights the superior efficacy of MLVs in contrast to inactivated vaccines.

IL-18, a proinflammatory cytokine belonging to the interleukin-1 family, was initially identified as an interferon-gamma-inducing factor (Nakamura K, et al., 1993, “Purification of a factor which provides a costimulatory signal for gamma interferon production,” Infect. Immun. 61: 64-70). IL-18 plays a crucial role in the host's response to influenza A virus. Studies involving chickens immunized with a recombinant fowl pox virus expressing H9 HA and chicken IL-18 suggest that IL-18 can enhance anti-HA responses when co-expressed (Chen HY, et al., 2011, “Immune responses of chickens inoculated with a recombinant fowlpox vaccine coexpressing HA of H9N2 avian influenza virus and chicken IL-18,” Antiviral Res. 91: 50-56). An eukaryotic expression plasmid encoding chicken IL-18 has been employed as a potential adjuvant in conjunction with an inactivated Newcastle disease vaccine, resulting in elevated antibody level responses and T and B lymphocyte proliferation compared to the group that received only the inactivated vaccine (Wang C, et al., 2015, “A eukaryotic expression plasmid carrying chicken interleukin-18 enhances the response to Newcastle disease virus vaccine,” Clin. Vaccine Immunol. 22: 56-64).

Consistent with these findings, the results shown here demonstrated that the inclusion of IL-18 elicited higher levels of HI titers and neutralizing antibodies compared to the same candidate without IL-18 (MLV-H9N2). Additionally, enhanced protection was observed in chickens vaccinated with MLV-H9N2-IL, as evidenced by reduced virus detection in OP swabs. This suggests that IL-18 may be playing a role in the host immune response, as observed for other infectious diseases (Wang 2015, Supra). Previous mouse studies have demonstrated that RAM and other MLV strategies induce limited virus replication and stimulate mucosal immunity. The present study focused on chickens, concentrated exclusively on evaluating whether the vaccine elicited detectable serum HI responses indicative of protection, as these responses are more readily assessed in the field.

Drinking water vaccination offers several advantages, including uniform and rapid delivery, reduced costs, and the potential for extensive mass vaccination, which is highly desirable for poultry species. Several studies have demonstrated the suitability of drinking water vaccination with MLVs, which have been successfully used and established for other important poultry diseases such as avian metapneumovirus, Fowl pox, infectious laryngotracheitis, and infectious bronchitis (Ganapathy K, et al., 2010, “Vaccination of commercial broiler chicks against avian metapneumovirus infection: a comparison of drinking-water, spray and oculo-oral delivery methods,” Vaccine 28:3944-3948; Hilbink F, et al., 1981, “Drinking water vaccination against infectious laryngotracheitis,” Can. J. Comp. Med. 45: 120-123). Live recombinant Newcastle disease vectored vaccines expressing the hemagglutinin of H9N2 and H5N1 have also been administered via drinking water vaccination and protected chickens against homologous challenges (Lee J, et al., 2023, “Live recombinant Newcastle disease virus vectored vaccine expressing the haemagglutinin of H9N2 avian influenza virus suppresses viral replication in chickens,” Avian Pathol. 52: 100-107; Veits J, et al., 2008, “Protective efficacy of several vaccines against highly pathogenic H5N1 avian influenza virus under experimental conditions,” Vaccine 26: 1688-1696)

In this study, whether MLV-H9N2-IL would be immunogenic and protective in chickens when administered via drinking water was investigated using prime-only or prime-and-boost strategies. After mixing the vaccine in distilled water, vaccine stability was analyzed at different temperatures over time. MLV-H9N2-IL at 10⁶ EID₅₀/mL remained stable during vaccine administration at 25° C., 30° C., and 40° C. This is particularly important because temperatures inside chicken houses can easily reach 40° C., which must be considered when delivering vaccines. MLV-H9N2-IL at 10⁴ EID₅₀/mL was stable at 25° C. but exhibited a rapid decline in titers when incubated at 30° C. and 40° C. Additionally, HI titers and neutralizing antibodies were readily detected after prime vaccination and increased after boost in a dose-dependent manner.

Both vaccine doses and strategies reduced viral loads after the homologous challenge in CL swabs, OP swabs, sinuses, trachea, and cloaca. Importantly, a prime-and-boost regimen at 10⁶ EID₅₀/mL induced sterilizing immunity in chickens, as evidenced by a complete absence of virus replication in all tissues mentioned above. The ability of MLV-H9N2-IL to completely prevent virus replication and shedding after challenge is highly significant, as these birds would not transmit the virus to new hosts, effectively breaking the transmission cycle of avian influenza. This study successfully demonstrates the generation and assessment of two MLVs using genome rearrangement. MLV-H9N2 and MLV-H9N2-IL are stable, grow similarly to an H9N2 WT virus in vitro, and exhibit an attenuated profile in vivo. Co-infection studies in vivo confirmed the lack of reassortment between MLV and WT virus segments. Both vaccine candidates are immunogenic and protective in chickens when administered via the EROC route. MLV-H9N2-IL administered via drinking water is also immunogenic and protective in chickens, providing an alternative to individual bird vaccination. Overall, this work provides novel insights into the development of vaccines against avian influenza carrying molecular markers and immunomodulators and their potential for mass vaccination applications in the field.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

The term “about” is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the recited value. For example, about 1.0 g means 0.9 g to 1.1 g.

The terms “individual,” “subject,” and “animal”, are used interchangeably herein, and refer to vertebrates that support a negative strand RNA virus infection, specifically influenza A virus infection.

Exemplary subjects may include vertebrates of importance to humans due to being endangered, being of economic importance such as those raised on farms for consumption by humans, and/or being of social importance such as animals kept as pets or in zoos. The methods and compositions of the present disclosure are particularly useful for warm-blooded vertebrates including, but not limited to, birds, Canidae, Cetacea, Felidae, Mustelidae, Rodentia, Equidae, Bovidae, Suidae, and Primates. In some embodiments the vertebrates are birds such as water fowl, chickens, or turkeys. In some embodiments the vertebrates are mammals such as pigs, horses, whales, dolphins, or humans. In some embodiments the vertebrates are humans. In some embodiments the vertebrates are chickens.

As used herein, the terms “polynucleotide sequence” and “nucleic acid sequence” are used interchangeably and refer to a character string representing the polynucleotide or nucleic acid. Unless otherwise indicated, a particular nucleic acid sequence optionally encompasses complementary sequences in addition to the sequence explicitly indicated. From any specified polynucleotide sequence, either the given nucleic acid or the complementary polynucleotide sequence (e.g., the complementary nucleic acid) can be determined.

The term “gene” is used herein broadly to refer to any nucleic acid associated with a biological function. Thus, genes include coding sequences and/or the regulatory sequences required for their expression. The term “gene” applies to a specific genomic sequence, as well as to a cDNA or an mRNA encoded by that genomic sequence.

Genes also include non-expressed nucleic acid segments that, for example, form recognition sequences for other proteins. Non-expressed regulatory sequences include “promoters” and “enhancers” to which regulatory proteins such as transcription factors bind, resulting in transcription of adjacent or nearby sequences. A “tissue specific” promoter or enhancer is one that regulates transcription in a specific tissue type or cell type, or types.

An “open reading frame” or “ORF” is a possible translational reading frame of DNA or RNA (e.g., of a gene), which is capable of being translated into a polypeptide. That is, the reading frame is not interrupted by stop codons. However, it should be noted that the term ORF does not necessarily indicate that the polynucleotide is, in fact, translated into a polypeptide.

As used herein, “Expression of a gene” or “expression of a nucleic acid” mean transcription of DNA into RNA (optionally including modification of the RNA, e.g., splicing), translation of RNA into a polypeptide (possibly including subsequent modification of the polypeptide, e.g., post-translational modification), or both transcription and translation, as indicated by the context.

As used herein, a “recombinant virus” is one which has been manipulated in vitro, e.g., using recombinant nucleic acid techniques to introduce changes to the viral genome, or a virus that is artificially generated.

As used herein, the terms “recombinant nucleic acid,” “recombinant segment,” and “recombinant polynucleotide” are used interchangeably and refer to a nucleic acid that has been altered in vitro. The sequence of a recombinant polynucleotide is not naturally occurring, or does not correspond to naturally occurring sequences, or that are not positioned as they would be positioned in the native genome.

By “pharmaceutical composition” is meant a composition that contains a recombinant mutant influenza gene segment of the invention, or a recombinant influenza virus of the invention, and that is suitable for administration to a subject. The pharmaceutical composition is suitable to prevent, treat, reduce, or ameliorate one or more influenza symptoms in the subject. For the purposes of this invention, pharmaceutical compositions include vaccines.

As used herein “diluent,” excipient,” “carrier,” and “adjuvant” are used interchangeably, and refer to a diluent, excipient, carrier, or adjuvant which is physiologically acceptable to the subject while retaining the therapeutic properties of the pharmaceutical composition with which it is administered. Physiologically acceptable diluents, excipients, carriers, or adjuvants and their formulations are known to one skilled in the art (see, e.g., U.S. Pat. No. 9,017,691; Chaudhari S. P., et al. 2012, Pharmaceutical Excipients: A Review,” IJAPBC Vol 1(1)). Reed S. G., et al. (2013, “Key Roles of Adjuvants in Modem Medicines,” Nature Medicine 19(12): 1597-1608) review adjuvants used in vaccines.

As used herein, the term “attenuated” used in conjunction with a virus, refers to a virus having reduced virulence or pathogenicity as compared to a non-attenuated counterpart, yet is still viable or live. Typically, attenuation renders an infectious agent, such as a virus, less harmful or virulent to an infected subject compared to a non-attenuated virus.

The terms “inoculated” and “vaccinated” are used interchangeably herein and refer to the act of introducing an influenza virus vaccine of the invention in birds.

The term “adjuvant”, as used herein, means a pharmacological or immunological agent that modifies the effect of other agents, such as a drug or immunogenic composition. Adjuvants are often included in immunogenic compositions to enhance the recipient's immune response to a supplied antigen.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Production of Recombinant Mutant Influenza a Viruses

Cells and Eggs. Madin-Darby canine kidney (MDCK) and human embryonic kidney 293T cells (HEK293T) were a kind gift from Robert Webster (St Jude Children's Research Hospital, Memphis, TN, USA). Cells were maintained in Dulbecco's Modified Eagles Medium (DMEM, Sigma-Aldrich, St Louis, MO, USA) containing 10% fetal bovine serum (FBS, Sigma-Aldrich), 1% antibiotic/antimycotic (AB, Sigma-Aldrich) and 1% L-Glutamine (Sigma-Aldrich). Cells were cultured at 37° C. and 5% CO₂. Specific pathogen-free embryonated chicken eggs (ECEs) used for virus propagation and stock titration were obtained from Charles Rivers (Wilmington, MA, USA).

Generation of reverse genetics plasmids. The genome rearrangement strategies for the production of MLVs have been previously described (Cardenas-Garcia S, et al., 2021, “FluB-RAM and FluB-RANS: Genome Rearrangement as Safe and Efficacious Live Attenuated Influenza B Virus Vaccines,” Vaccines (Basel) 9; Pena L, et al., 2013, “Influenza viruses with rearranged genomes as live-attenuated vaccines,” J. Virol. 87: 5118-5127. The reverse genetics backbone of the prototypical LPAIV strain A/guinea fowl/Hong Kong/WF10/1999 (H9N2) -G1/h9.4.1 lineage—has been previously described (Song H, et al., 2007 “A new generation of modified live-attenuated avian influenza viruses using a two-strategy combination as potential vaccine candidates,” J. Virol. 81: 9238-9248.). To generate the plasmid pDP-PB1M2-WF10, the plasmid pDP-PB1WF10 was modified to preserve packaging signals in gene segment 2 (Liang Y, et al., 2008, “Mutational analyses of packaging signals in influenza virus PA, PB1, and PB2 genomic RNA segments,” J. Virol. 82: 229-236) and carrying a chimeric PB1 ORF with a C-terminal tag consisting of a Gly-Gly-Gly-Gly-Ser (G4S; set forth in SEQ ID NO: 1) spacer, the Thosea asigna virus 2A protease (2a Tav) and the M2 Open Reading Frame (ORF). The pDP-M1ΔM2 was generated from the plasmid pDP-MWF10 by site-directed mutagenesis to carry multiple early stop codons in the M2 ORF to prevent its expression. The pDP-DRPAVIAN-HA-ck/EGY carries a chimeric HA segment derived from the H9N2 strain A/chicken/Egypt/A15068/2018 (ck/EGY) carrying a unique 8 amino acids-long sequence, including the peptide sequence Asp-Arg-Pro-Ala-Val-Ile-Ala-Asn (DRPAVIAN; set forth in SEQ ID NO: 2) placed downstream of the HA signal peptide sequence but upstream of the mature HA ORF. To generate the plasmid pDP-NAckIL18, the plasmid pDP-NAWF10 was digested with BamHI and HindIII and then a synthetic fragment encoding the mature protein sequence of chicken IL-18 (ckIL18, GenScript, Piscataway, NJ, USA) was subcloned at the C-terminus in frame with the NA ORF. Plasmids were propagated in TOP-10 E. coli cells (ThermoFisher, Waltham, MA, USA) and purified using the QIAGEN Plasmid Maxi Kit (Qiagen, Gaithersburg, MD, USA). Plasmid sequences were confirmed by Sanger Sequencing (Psomagen, Rockville, MD, USA).

Generation of MLVs by reverse genetics. Recombinant viruses were rescued by reverse genetics using the 8-plasmid system in co-cultured HEK293T and MDCK cells in 6-well plates following Hoffmann E, et al. (2002, “Eight-plasmid system for rapid generation of influenza virus vaccines,” Vaccine 20:3165-3170). The MLV-H9N2 virus was generated from the following combination of plasmids: pDP-PB2-WF10, pDP-PB1M2-WF10, pDP-PAWF10, pDP-DRPAVIAN-HA-ck/EGY, pDP-NPWF10, pDP-NAWF10, pDP-M1ΔM2-WF10, pDP-NSWF10. For the generation of MLV-H9N2-IL, the plasmid pDP-NAckIL18 was used instead of pDP-NAWF10. On transfection day, 1 μg of each plasmid was mixed with the TransIT-LT1 transfection reagent (Mirus Bio LLC, Madison, WI, USA) in a ratio of 1 μg plasmid DNA/2 μL of transfection reagent in a final volume of 1 mL of Opti-MEM® media (Fisher Scientific, Hampton, NH, USA). The mixture was incubated for 45 minutes and then used to overlay the 293T/MDCK cells overnight. The next day, the transfection mixture was replaced with fresh Opti-MEM® media containing 1% AB (Sigma-Aldrich). At 24 and 72 hours post-transfection 1 μg/mL of tosylsulfonyl phenylalanyl chloromethyl ketone (TPCK)-treated trypsin (Worthington Biochemicals, Lakewood, NJ, USA) was supplemented to the cells. Viral stocks were generated in 10-day-old specific pathogen-free (SPF) eggs. Allantoic fluids were harvested at 48 hours post-infection (hpi), centrifuged, aliquoted, and stored at −80° C. These stocks constitute the first passage in eggs (E1). Viruses were titrated by tissue culture infectious dose 50 (TCID₅₀) and egg infectious dose 50 (EID₅₀). Virus titers were established by the Reed and Muench method (Reed L J and Muench H, 1938, “A simple method of estimating fifty percent endpoints,” Am. J. Epidemiol. 27:493-497.). All viruses used in this study were confirmed by Sanger sequencing (Psomagen).

### Example 2

### Recombinant Virion Testing

The genome stability, in vitro growth kinetics, co-infection in vitro, and sequencing of the virions prepared herein were tested.

Genome stability of MLVs. The MLVs were serially diluted 10-fold in phosphate-buffered saline (PBS) containing 1% AB (Sigma-Aldrich); 100 μL of 3 different dilutions were inoculated in 10-day-old SPF eggs. Eggs were incubated at 35° C. for 48 hours, placed at 4° C. overnight and then allantoic fluid from each inoculated egg was collected to perform hemagglutination assays using 0.5% chicken red blood cells. For each MLV, the dilution with the highest HAU was harvested, centrifuged, aliquoted, and stored at −80° C. Five serial passages were performed following the same procedure described above. The MLVs harvested from egg passage 5 (E5) were used to generate a viral stock. Viral RNA extraction of both MLVs (E1 and E5) was carried out using the QIAquick® Viral RNA Mini Kit (Qiagen, Germantown, MD, USA). Viral RNA was amplified by RT-PCR using SuperScript III One-Step RT-PCR Platinum Taq HiFi DNA polymerase (ThermoFisher). The PB1 segment was amplified using the forward primer PB1-2068F (5′-TTCTTGAGGATGAACAGATGTACCAGAAGTGC-3′; set forth in SEQ ID NO: 3) and reverse primer Bm-PB1-2341R-eq (5′-ATATCGTCTCGTATTAGTAGAA ACAAGGCATTTTTTCATG-3′; set forth in SEQ ID NO: 4). The HA segment was amplified using the forward primer H9HA-1F (5′-CTAGCAGTTAACCGGAGTACTGG-3′; set forth in SEQ ID NO: 5) and the reverse primer H9HA657-693Rev (5′-GACAAGGGGCCTTGGCCCTA TCACTGGTTTGAAGGTC-3′; set forth in SEQ ID NO: 6). The NA segment was amplified using the forward primer WF10NA118F (5′-GATTCGCGCTCTGGTTATGA-3′; set forth in SEQ ID NO: 7) and the reverse primer N2Hu_1463R (5′-AGTAGAAACAAGGAGTTTTTTC-3′; set forth in SEQ ID NO: 8). The amplicons were resolved on an agarose gel, purified using the QlAquick® Gel Extraction Kit (Qiagen), and analyzed by Sanger sequencing (Psomagen).

Growth kinetics in vitro. Six-well plates were seeded with 3×10⁵ MDCK cells per well the day before virus inoculation. Cells were inoculated with either H9N2 WT, MLV-H9N2, MLV-H9N2-IL, or H9N2att following Song H, et al. (2007, Supra) at a multiplicity of infection (MOI) of 0.01. Upon virus inoculation, cells were incubated for 15 minutes at 4° C. and then for 45 minutes at 37° C. Subsequently, cells were washed twice with 1 mL of PBS (Life Technologies, Carlsbad, CA, USA) and supplemented with 2 mL of Opti-MEM® (Fisher Scientific) containing TPCK-trypsin (Worthington Biochemicals, Lakewood, NJ, USA) and 1% AB (Sigma-Aldrich) and further incubated at 37° C. and 5% CO₂. Supernatants were collected at 0, 12, 24, 48, 72, and 96 hpi and used to quantify the levels of viral replication by real-time quantitative reverse transcriptase PCR (RT-qPCR). For this purpose, viral RNA (vRNA) was extracted using the MagMAXTM-96 viral RNA Isolation kit (ThermoFisher). The quantification of vRNA was based on the Influenza A matrix gene using the forward primer M+25 (5′-AGATGAGTCTTCTAACCGAGGTCG-3′; set forth in SEQ ID NO: 9) and the reverse primer M-124 (5′-TGCAAAAACATCTTCAAGTCTCT G-3′; set forth in SEQ ID NO: 10) along with the qPCR probe M+64 (5′-GGCCCCCTCAAAGC CGA-3′; set forth in SEQ ID NO: 11) labeled at the 3′ end with the fluorophore FAM, and at the 5′ end with the quencher TAMRA. Experiments were carried out in a QUANTSTUDIO® 3 (Applied Biosystem, Foster City, CA, USA) using qScript® XLT One-Step RT-qPCR ToughMix® Quantabio® (ThermoFisher). Standard curves to correlate quantitative PCR crossing-point values with virus titers were made using a 10-fold dilution from a virus stock of known titer. Virus titers after qPCR were expressed as log₁₀ TCID₅₀ equivalents/mL (log₁₀ TCID₅₀ equivalent/mL).

Western blotting. Confluent MDCK cells were inoculated with the MLVs (MLV-H9N2 and MLV-H9N2-IL) at an MOI of 1 or 10 for 1 hour at 37° C. Following inoculation, the inoculum was removed and replaced with fresh Opti-MEM® culture media (Fisher Scientific). Infected cells were incubated at 37° C. for 10 hours. Moreover, confluent HEK293T cells were transfected with 10 μg of pCAGGS-Furin2A-IL-18-HIS for 48 hours at 37° C. After incubation, the media was removed from the cells and cold Radio-ImmunoPrecipitation Assay (RIPA) buffer (ThermoFisher) was used to lyse the cells. Quantification of total proteins was performed using BCA protein assay kit (ThermoFisher). Then, 150 μL of supernatant was mixed with 150 μL of Laemmli buffer containing mercaptoethanol (Bio-Rad, Hercules, CA, USA), followed by boiling for 7 minutes, and brief sonication. Proteins were separated on a 12% sodium dodecyl sulfate-polyacrylamide gel electrophoresis (SDS-PAGE) gel and were transferred to nitrocellulose membranes (Bio-Rad) for immunoblot analysis. Membranes were blocked in 5% molecular-grade nonfat dry milk overnight on shaker at 4° C. Then, membranes were incubated with a mouse primary antibody against chicken IL-18 (Kerafast, Inc., Boston, MA, USA) or a mouse primary antibody against glyceraldehyde-3-phosphate dehydrogenase (GAPDH) (Fisher Scientific) for 2 hours shaking at room temperature. After washing, the membranes were incubated with the respective secondary antibodies diluted in 5% molecular-grade nonfat dry milk: goat anti-mouse secondary antibody (Fisher Scientific) and anti-mouse IgG-horseradish peroxidase (HRP) conjugate (Fisher Scientific) for 1 hour shaking at room temperature. Finally, the membranes were washed and imaged through a chemiluminescent reaction using West Femto ultra sensitive enhanced chemiluminescent (ECL) substrate (ThermoFisher Scientific). The ChemiDoc MP Imaging System for imaging and analyzing gels and western blots (Bio-Rad) was employed to visualize the membranes and capture the images.

Co-infection studies in vitro. MDCK cells were seeded in 6-well plates at a ratio of 3×10⁵ cells per well. The next day, cells were co-infected with a mixture of either MLV-H9N2 or MLV-H9N2-IL and A/Chicken/Tunisia/812/2012 (H9N2) (ck/Tun) at the ratio 10MLV:1WT as follows: MLV-H9N2 (MOI 0.01)+ck/Tun (MOI 0.001) and MLV-H9N2-IL (MOI 0.01)+ck/Tun (MOI 0.001). The co-infection was performed with 500 μL of each mixture for 15 minutes at 4° C. and then 45 minutes at 37° C. Subsequently, the inoculum was removed, and cells were washed twice with 1 mL of PBS (Life Technologies, Carlsbad, CA, USA). Finally, 2 mL of Opti-MEM® (Fisher Scientific) supplemented with TPCK-trypsin (Worthington Biochemicals, Lakewood, NJ, USA) and 1% AB (Sigma-Aldrich, St. Louis, MO, USA) was added to the cells. Cells were incubated at 37° C. and 5% CO₂. Supernatants collected at 72 hpi were used for limiting dilution assays as described elsewhere (Kimble J B, et al., 2014, “Alternative reassortment events leading to transmissible H9N1 influenza viruses in the ferret model,” J. Virol. 88:66-71) to isolate cloned populations. Briefly, a 96-well plate was seeded with 1.5×10⁴ MDKC cells/well. Cells were infected with 8, 10-fold serial dilutions of each virus combination. After 72 hours at 37° C., supernatants were collected from wells infected with the most diluted sample displaying cytopathic effect (CPE) and then used for the second round of infection by limiting dilution. Supernatants from the second round of infection were collected at 72 hours and used for sequencing.

Next-Generation Sequencing (NGS). The vRNA from each sample was extracted using the MagMAX-96 viral RNA Isolation kit (ThermoFisher). Multi-Segment RT-PCR (MS-RTPCR) was performed as previously described (Zhou B, et al., 2009, “Single-reaction genomic amplification accelerates sequencing and vaccine production for classical and Swine origin human influenza a viruses,” J. Virol. 83:10309-10313.). Briefly, 2.5 μL of vRNA was used as a template in a 25 μL MS-RTPCR reaction (SuperScript III One-Step RT-PCR Platinum Taq HiFi DNA polymerase (ThermoFisher)) using primers Optil-F1 5′-GTTACGCGCCAGCAAAAGC AGG-3′, set forth in SEQ ID NO: 12; Optil-F2 5′-GTTACGCGCCAGCGAAAGCAGG-3′, set forth in SEQ ID NO: 13; and Optil-R1 5′-GTTACGCGCCAGTAGAAACAAGG-3′, set forth in SEQ ID NO: 14. The final product was analyzed in 1% agarose gel to verify whole genome amplification (Ferreri L M, et al., 2019, “Improved detection of influenza A virus from blue-winged teals by sequencing directly from swab material,” Ecol. Evol. 9:6534-6546). MS-RTPCR amplicons were sequenced using the Illumina platform as described elsewhere (Ortiz L, et al., 2023, “Blue-Winged Teals in Guatemala and Their Potential Role in the Ecology of H14 Subtype Influenza a Viruses,” Viruses 15: 483). Briefly, products were purified using 0.45× of AGENCOURT® AMPure XP Magnetic Beads (Beckman Coulter, Brea, CA, USA) and eluted in HyClone® molecular biology water (Genesee Scientific, San Diego, CA, USA). The concentration of the samples was determined using a Qubit® buffer kit (ThermoFisher) in a Qubit® 3.0 fluorometer (ThermoFisher) and normalized to 0.2 ng/μL. Indexes were added via tagmentation using the Nextera® XT DNA library preparation kit (Illumina, San Diego, CA, USA). The reaction was set as 40% of the suggested final volume. Next, samples were purified using 0.7× of Agencourt® AMPure® XP Magnetic Beads and analyzed on a Bioanalyzer using a High Sensitivity DNA kit (Agilent, Santa Clara, CA, USA). Libraries were pooled and normalized to 0.5 nM. After denaturation, the final loading concentration of the pooled libraries was 14 pM. Libraries were sequenced using a MiSeq® Reagent Kit V2, 300 cycles (Illumina, San Diego, CA, USA). Genome assembly was performed using a pipeline previously described (Mena I, et al., 2016, “Origins of the 2009 H1N1 influenza pandemic in swine in Mexico’” eLife 5:e16777). For co-infection studies, FASTQ files generated were used to map the proportion (%) of NGS reads that matched either the MLV- or WT-specific segment. For unmodified segments (PB2, PA, NP, NS), the FASTQ file was used to map the reads to the whole WT or MLV segment, and the proportion was established based on the number of reads for the specific segment (WT or MLV) divided by the total number of reads for the given sample. To distinguish between WT or MLV-modified segments (PB1, HA, NA, and M), NGS reads were mapped to the junctions (PB1-M2; DRP-HA; NA-ckIL-18; M2esc).

Preparation of WIV-adj vaccine control. Reverse genetics was used to prepare a virus carrying the H9 HA of ck/EGY and the NA of the WF10 virus in the background of the laboratory-adapted strain A/Puerto Rico/08/34 (H1N1). Upon virus rescue, stocks were grown in ECE. To inactivate the virus, allantoic fluid containing the live virus was mixed with Binary Ethyleneimine 0.1M solution and incubated at 37° C. in a T175 flask (ThermoFisher). After 13 hours, this solution was added to a new T175 flask and incubated for 7 hours at 37° C. Next, 1M sodium thiosulfate solution was added to the BEI-treated virus stock, mixed thoroughly and the pH was adjusted to 7.2. An aliquot of the inactivated virus was inoculated into ECE and MDCK cells to confirm the inactivation process. On the day of vaccination, the vaccine emulsions were prepared by mixing Montanide® ISA71 VG (Seppic, Fairfield, NJ, USA) with the inactivated virus at a 70:30 ratio Montanide:Virus at 512 HAU per dose.

### Example 3

### In Vivo Studies

Vaccine-challenge studies in chickens. Studies were approved by the Institutional Animal Care and Use Committee (IACUC) at the University of Georgia (Animal Use Protocol A2022 09-001-Y1-A5) to be performed under ABSL2 conditions. SPF White Leghorn embryonated eggs (Charles River Laboratories International Inc., Wilmington, MA, USA) were hatched and chicks raised for 2 weeks. In the first experiment, 4 groups of 2-week-old chickens were established and inoculated through the eyes and the respiratory (nasal and tracheal), oral, and cloacal routes (EROC) with either 1×10⁶ EID₅₀/chicken of MLV-H9N2 (n=30), MLV-H9N2-IL (n=30) or control PBS (mock vaccinated group; n=35) or via the subcutaneous route with 512 HAU/chicken of the WIVadj-H9N2 vaccine (n=30). Chickens were boosted 2 weeks after prime with the corresponding homologous vaccine using the same routes. Two weeks after the boost, chickens were challenged with 1×10⁸ EID₅₀/chicken of the homologous virus via EROC route. The challenge virus was a recombinant virus encoding the wild-type H9 HA of ck/EGY in the background of the wild-type WF10 strain (ck/EGY/WF10). In the second experiment, 2-week-old SPF White Leghorn chickens were vaccinated via drinking water with the MLV-H9N2-IL vaccine at either 10⁴ EID₅₀/mL (n=30) or 10⁶ EID₅₀/mL (n=30) in a prime-only or prime and boost 2 weeks apart. A subset of chickens (n=30) was administered water only. The MLV-H9N2-IL virus vaccine was diluted in distilled sterile water with Vac-Pac Plus (Best Veterinary Solutions, Columbus, GA, USA) to neutralize residual chlorine or salts in the water and stabilize the pH. Before vaccine administration, chickens were deprived of water for 6 hours. Drinking water vaccination proceeded for 2 hours with the MLV-H9N2-IL virus vaccine administered using bell drinkers. Two weeks after prime, half of the chickens (n=15) in each group were boosted with the same doses as the prime. Two weeks after the boost or four weeks after prime, chickens were challenged with 1×10⁸ EID₅₀/chicken of the ck/EGY/WF10 virus via EROC route as described previously.

Transmission of MLV-H9N2 and MLV-H9N2-IL viruses and reassortment in vivo in chickens. Chickens (2-week-old) were inoculated with 1×10⁶ EID₅₀/chicken via the EROC route with either the MLV-H9N2 (n=4) or MLV-H9N2-IL (n=4). At 24 hpi, directly inoculated chickens were moved into a clean isolator that contained naïve direct contact chickens (n=4) for each respective group. Oropharyngeal (OP) and cloacal (CL) swabs were collected every day until 6 days post-inoculation (dpi) and 6 days post-contact. Viral loads were analyzed by RT-qPCR as described previously. In the fourth experiment, 2-week-old chickens were coinfected with the MLV-H9N2 (n=4) or MLV-H9N2-IL (n=4) at 1×10⁶ EID₅₀/chicken and Ck/Tun wild-type virus at 1×10⁵ EID₅₀/chicken (ratio 10MLV:1WT). Oropharyngeal (OP) and cloacal (CL) swabs were collected every day until 5 days days-post infection (dpi) and directly sequenced by NGS as described above.

Vaccine Stability in Drinking Water. The MLV-H9N2-IL virus vaccine diluted in sterile water with Vac-Pac Plus® was incubated at different temperatures in a thermocycler: 25° C., 30° C., 40° C., and 50° C. Time points were collected at 0, 1, 2, and 24 hours after vaccine preparation. All time points were titrated by TCID₅₀, and vaccine stability expressed as the amount of infectious virus present at the specific time point with virus titers established by the Reed and Muench method (Supra).

Nucleoprotein Enzyme-Linked Immunoassays. Blood was collected from a subset of chickens per group 12 days post-vaccination (dpv), and 12 days post-boost (dpb). The levels of nucleoprotein (NP) antibodies were determined using the IDEXX® AI MultiS-Screen Avian Influenza Virus Antibody Test kit (IDEXX Laboratories Inc., Westbrook, ME, USA). The presence or absence of antibodies was determined by relating the absorbance values at 650 nm to the negative control mean. A threshold for positive samples of 0.6 was established following the manufacturer's recommendations.

Hemagglutination Inhibition (HI) assays. Blood was collected from a subset of chickens per group 12 days dpv, and 12 dpb. In addition, blood was collected from a subset of chickens 26 dpv for the second experiment—drinking water vaccination—in the prime-only group. Sera samples were analyzed by hemagglutination inhibition assay as described elsewhere (Ciceres C J, et al., 2021, “Development of a Novel Live Attenuated Influenza A Virus Vaccine Encoding the IgA-Inducing Protein,” Vaccines (Basel) 9(7): 703) to detect the presence of neutralizing antibodies. Briefly, sera were treated twice with 50% chicken red blood cells (RBCs) and diluted 1:10 in PBS. Fifty L of this dilution was added to the first column of a 96-well plate and 2-fold serially diluted. Then, sera were mixed with 4 HAU/25 L of the homologous virus. The virus/sera mixture was incubated for 30 minutes at room temperature and the HI activity was determined after 30 minutes of incubation with 0.5% of chicken RBCs. HI titers below 10 were arbitrarily assigned a value of 10.

Virus neutralization assays. Blood was collected from a subset of chickens per group 12 dpv, and 12 dpb. In addition, blood was collected from a subset of chickens 26 dpv for the second experiment—drinking water vaccination—in the prime-only group. Serum samples were treated twice with 50% RBCs as described previously. Serum samples were used to perform virus neutralization assays as described elsewhere (Ciceres C J, et al., 2021, Supra) but modified to rely on Nanoluciferase activity (VNLuc). Briefly, a homologous H9N1 influenza virus was generated in the PR8 backbone carrying a Nanoluciferase gene downstream of the PB1 gene. Sera samples were serially diluted in a 96-well plate and incubated with 100 TCID₅₀ of the homologous virus. The serum/virus mixture was incubated for 1 hour at 37° C. and then overlayed on MDCK cells for 15 minutes at 4° C. and 45 minutes at 37° C. The mixture was removed, cells were supplemented with Opti-MEM®-AB and TPCK and incubated for 48 hours as described previously. VNluc titers were read out using the Nano-Glo Luciferase Assay System (Promega, Madison, WI, USA) with a Victor X3 multilabel plate reader (PerkinElmer, Waltham, MA, USA).

Virus Titration. OP and CL swabs were collected on 3 and 5 dpv to determine vaccine viral replication in the intestinal and respiratory tracts. In addition, OP and CL swabs were collected at 1-, 3-, 5-, and 7-days post-challenge (dpc). Briefly, swab samples were spun down, 10-fold serially diluted, and directly inoculated into a 96-well plate seeded with 1.5×10⁴ MDCK cells/well. Sinuses, trachea, lungs, pancreas, and cloaca were collected on days 3 post-challenge to analyze vaccine protection after homologous challenge. Tissue homogenates were generated using the Tissue Lyzer II (Qiagen, Hilden, Germany). Briefly, 1 mL of PBS-AB was added to each sample with Tungsten carbide 3 mm beads (Qiagen, Hilden, Germany) in the tubes. Samples were homogenized for 10 minutes and then centrifuged at 15,000 g for 10 minutes Supernatants were collected, aliquoted, and directly inoculated into a 96-well plate seeded with 1.5×10⁴ MDCK cells/well. All samples were titrated by TCID₅₀ and virus titers were established by the Reed and Muench method (Supra). Alternatively, samples were also titrated by RT-qPCR. For this purpose, vRNA was extracted using the MagMAXTM-96 viral RNA Isolation kit (ThermoFisher) and the quantification of vRNA was based on the Influenza A matrix gene as described above.

Graphs/Statistical Analyses. All data analyses and graphs were performed using GraphPad® Prism@software version 9 (GraphPad Software Inc., San Diego, CA, USA). Ordinary one-way ANOVA was performed to calculate P values followed by Tukey's multiple comparison test. A P value below 0.05 was considered significant.

### Example 4

### Vaccine Candidate Results

The MLV-H9N2 and MLV-H9N2-IL vaccine candidates, featuring rearranged genomes and modifications in HA and NA segments, exhibited stability after serial passage in vitro. The viruses were developed based on the prototypical WF10 H9N2 strain with a rearranged genome (RAM), involving the placement of the M2 open reading frame (ORF) downstream of PB1 and the introduction of stop codons in the original M2. In the RAM backbone, M2 is predicted to be co-translationally separated from PB1 via the action of the Thosea asigna 2A protease (2ATav) sequence encoded between the PB1 and M2 ORFs. The MLV-H9N2 and MLV-H9N2-IL candidates carry a segment 4 (HA) encoding a 8 amino acid long sequence containing the unique DRPAVIAN peptide sequence placed downstream of the HA signal peptide sequence but upstream of the mature HA ORF. The modified HA segment was derived from the sequence of a chicken H9N2 isolate identified in Egypt in 2018 (ck/EGY). The DRPAVIAN peptide is predicted to be co-translationally removed from the HA allowing bona fide expression of HA trimers on the virus surface. The DRPAVIAN peptide sequence is unique to the MLV-H9N2 and MLV-H9N2-IL candidates and does not exist in any known sequence of any living organism available in public databases. The unique sequence is expected to reduce the HA fitness for reassortment and can be used as a differentiation method compared to wild-type HA segments.

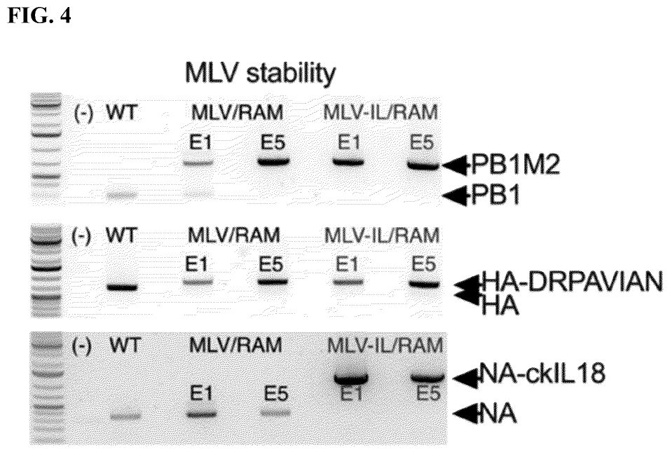

In the MLV-H9N2-IL candidate, the ckIL18 sequence was added to the C-terminus of the N2 NA, separated by a spacer released during translation via 2ATav activity. The MLV-H9N2 and MLV-H9N2-IL virus vaccine candidates were initially rescued in embryonated SPF chicken eggs and labeled as egg passage 1 (E1). The E1 MLV-H9N2 and MLV-H9N2-IL virus stocks were serially passaged five times in eggs (E5) to monitor the stability of genome modifications. RT-PCRs were performed targeting each modified segment from the E1 and E5 virus passages. As seen in FIG. 4, the sizes of the PCR products were maintained in both E1 and E5 passages which were bigger than those obtained from the unmodified segment controls. Genome stability was further confirmed by Sanger sequencing with no discernible mutations in any of the modified segments. FIG. 4 shows the RT-PCR fragment profiles of MLVs before serial passages in eggs (E1) or after serial passages in eggs (E5) targeting the PB1-M2 (top), HA-DRPAVIAN (middle), and NA-IL-18 (bottom). Wild-type segments were included as positive controls to demonstrate the differences in size between modified and non-modified segments. Note that band sizes are similar in E1 and E5 for all segments and vaccine candidates analyzed.

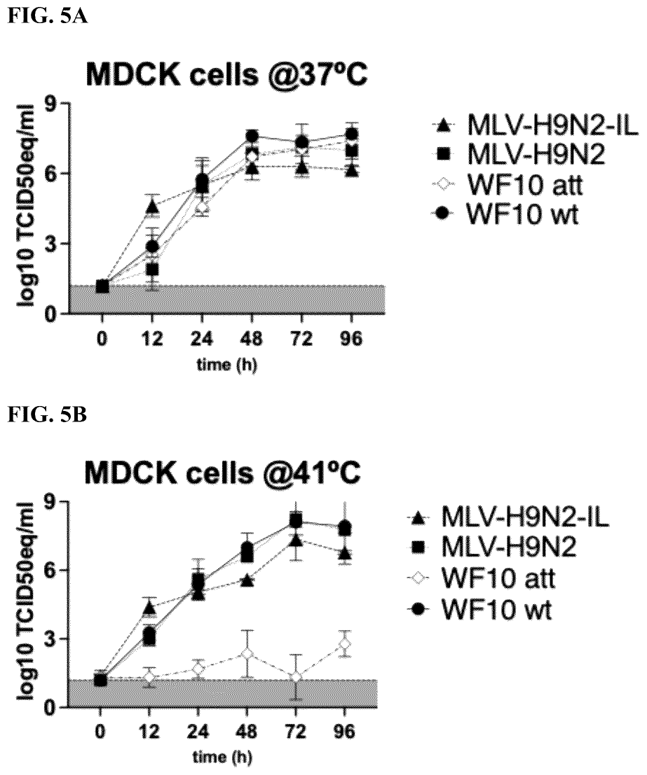

To determine whether genome rearrangements affect virus growth, MDCK cells were infected at an MOI of 0.01 and incubated at 37° C. or 41° C. for 96 hours. FIG. 5A and FIG. 5B present growth kinetics profiles of MLVs in MDCK cells at 37° C. and 41° C. RAM viruses grow at 41° C., unlike the temperature-sensitive control (WF10att). MDCK cells were inoculated with the MLV-H9N2, MLV-H9N2-IL, H9N2 WT, and WF10att (at an MOI of 0.01 and incubated at 37° C. and 41° C. for 96 hours. Samples were collected at 0, 12, 24, 48, 72, and 96 hours post-infection (hpi) and titrated by RT-qPCR. Virus titers are shown as the mean Log₁₀ TCID₅₀ equivalent/mL. Samples with undetected virus titers were assigned the limit of detection value (0.699 Log₁₀ TCID₅₀ equivalent/mL). Data analysis and graphs were prepared using Prism v9. As seen in FIG. 5A and FIG. 5B, compared with the H9N2 WT virus, the MLV-H9N2 and MLV-H9N2-IL viruses showed similar virus replication at 37° C., with minor differences at 12 hpi but similar replication levels at 24, 48, 72, and 96 hpi. At 41° C., both MLVs showed similar virus replication compared to the H9N2 WT virus. In contrast, the control H9N2att showed impaired replication at 41° C. but not 37° C. due to the introduction of temperature-sensitive mutations (Song H, Supra). The E4 passage virus was used to prepare an E5 stock which was used for vaccine studies. The E5 stock of the MLV-H9N2 reached titers of 3.16×10⁸ EID₅₀/mL while the MLV-H9N2-IL reached titers of 6.81×10⁷ EID₅₀/mL. Overall, these studies showed that the MLV-H9N2 and MLV-H9N2-IL viruses are stable and grow to high titers, characteristics that make them potentially suitable vaccine candidates.

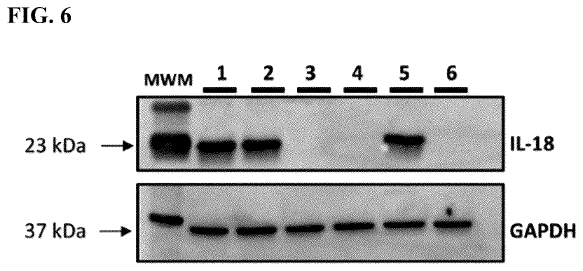

To demonstrate that IL-18 is being expressed from the MLV-H9N2-IL virus, MDCK cells were inoculated with the MLV-H9N2-IL and MLV-H9N2 viruses at low (1) and high (10) MOI. Cell lysates from HEK293T cells transfected with a plasmid expressing the N2WF10-Furin-2A-IL-18 product were used as positive control. FIG. 6 shows IL-18 expression from the MLV-H9N2-IL virus by western blot. MDCK cells were inoculated as follows: lane 1 (MLV-H9N2-IL; MOI 1), lane 2 (MLV-H9N2-IL; MOI 10), lane 3 (MLV-H9N2; MOI 1), lane 4 (MLV-H9N2; MOI 10). Lane 5, protein lysates of HEK293T cells transfected with the control expression plasmid pCAGGS expressing N2WF10-Furin-2A-IL-18. Lane 6, negative control cell lysates. The arrows indicate the predicted molecular weight of chicken IL-18 (23 KDa) and the host cellular protein glyceraldehyde-3-phosphate dehydrogenase (GAPDH; 37 KDa), which is shown as a gel loading control. MWM is the molecular weight marker. As seen in FIG. 6, IL-18 expression was detected from the MLV-H9N2-IL virus, regardless of the MOI employed. As expected, no IL-18 expression was seen from the control MLV-H9N2 virus.

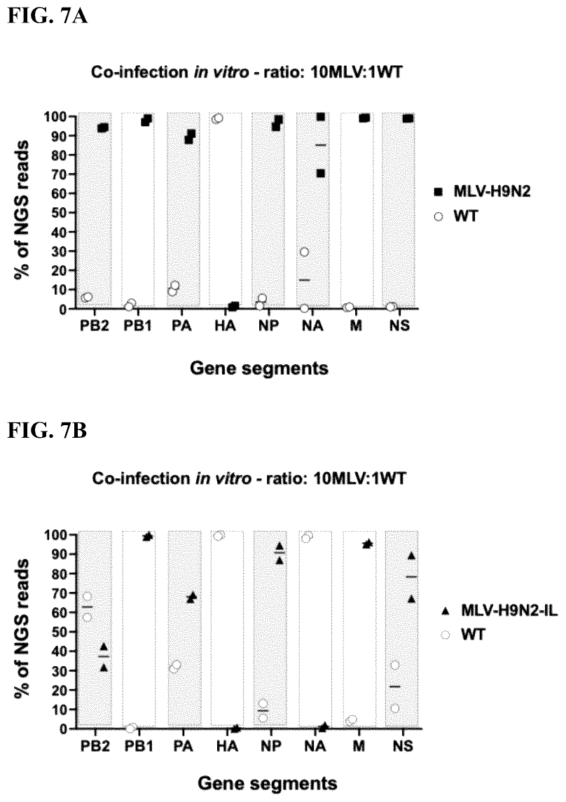

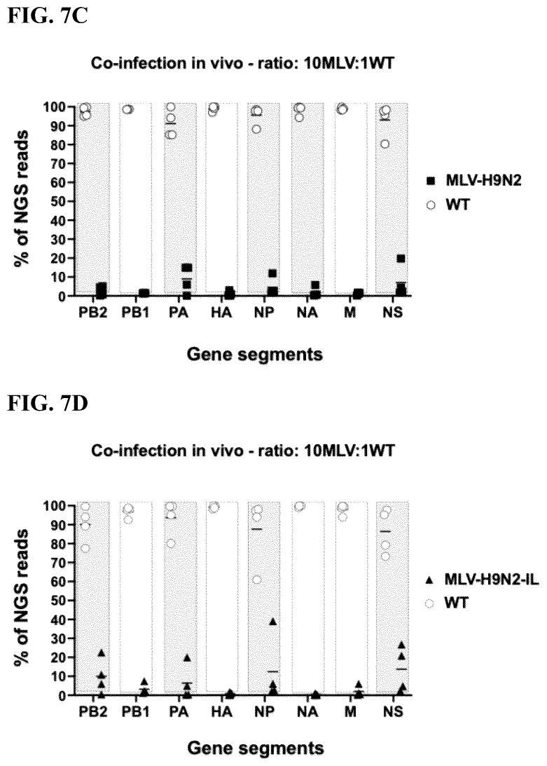

Modifications made to the HA and NA segments affected their fitness for reassortment both in vitro and in vivo. Both MLVs exhibited a fitness disadvantage compared to the wild-type (WT) virus in vivo. Co-infection studies in MDCK cells were conducted to assess the reassortment potential of the modified segments in the vaccine virus. These studies involved co-infecting cells with either the MLV-H9N2 or MLV-H9N2-IL strain and a prototypic WT H9N2 strain (ck/Tun) at a ratio of 10:1 (MLV:WT). Supernatants collected at 72 hpi were used for two rounds of limiting dilution to isolate single variants that were sequenced by NGS. FASTQ files were used to calculate the proportion of next-generation sequencing (NGS) reads that matched MLV or WT for each specific segment. As seen in FIG. 7A and FIG. 7B, the NGS data revealed that the DRPAVIAN modification rendered the modified HA less fit than the WT HA from the ck/Tun strain. None of the progeny virus clones contained the DRPAVIAN-modified HA segment. Similarly, the ckIL18-modified NA was largely outcompeted by the WT NA from the ck/Tun strain during co-infection in vitro. In contrast, the modified PB1-M2 and M1ΔM2 segments did not exhibit a significant fitness disadvantage and behaved similarly to unmodified gene segments. These in vitro studies indicated that the modifications introduced into the HA and NA segments, but not those in PB1 and M, affected segment fitness.

To further evaluate reassortment potential in vivo, co-infection studies were conducted in two-week-old chickens using a 10:1 (MLV:WT) ratio to favor reassortment. OP swabs collected on day 5 post-inoculation (dpi) were sequenced using NGS. The reassortment data upon co-infection with MLV-H9N2 is shown in FIG. 7C, and the reassortment data upon co-infection with MLV-H9N2-IL is shown in FIG. 7D. Consistent with the in vitro findings, both MLV-H9N2 and MLV-H9N2-IL exhibited a clear fitness disadvantage compared to the WT virus in vivo and were outcompeted. Importantly, reassortment between the MLV viruses and the WT virus was not observed in vivo. Interestingly, the modified PB1-M2 and M1ΔM2 segments, which did not exhibit a significant fitness disadvantage in vitro, demonstrated reduced fitness in vivo, along with all other gene segments. Collectively, these data demonstrate that the MLVs are safe and possess a fitness disadvantage compared to the WT virus. In the figure, filled squares (n) denote MLV-H9N2; empty circles (∘) denote WT, filled triangles (▴) denote MLV-H9N2-IL; empty circles (∘) denote WT. CLGray rectangles represent unmodified MLV segments while white rectangles represent modified MLV segments.

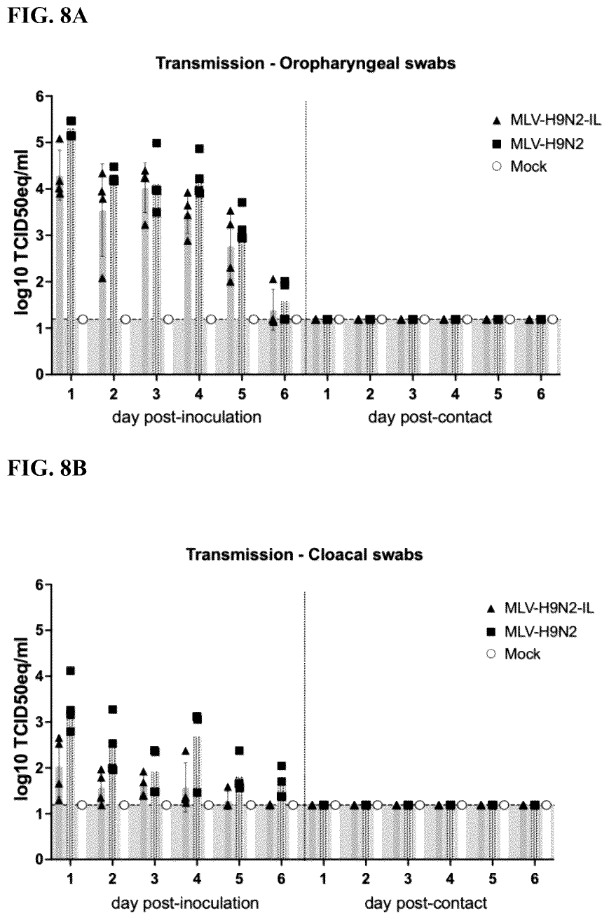

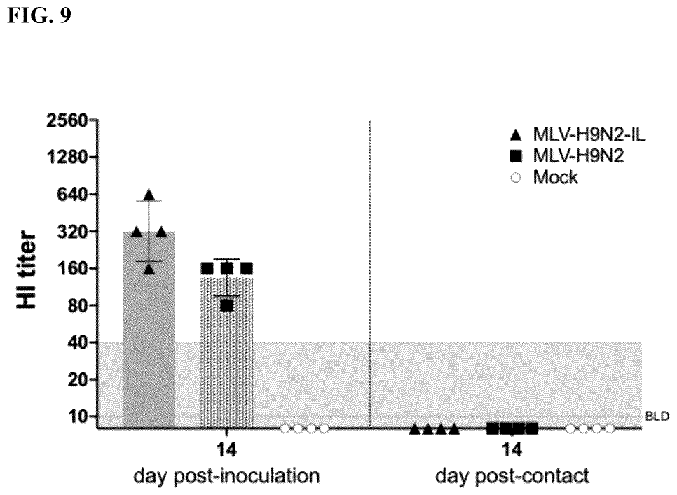

To evaluate the potential for transmission of MLV-H9N2 and MLV-H9N2-IL viruses in vivo in chickens, their ability to transmit to naïve direct contact chickens was assessed. Two-week-old chickens were inoculated with either MLV-H9N2 or MLV-H9N2-IL virus via the EROC route at a dose of 1×10⁶ EID₅₀/chicken. Naïve direct contact chickens were introduced 24 hpi, and OP swabs and CL swabs were collected (n=4/group/day) every day from 1 to 6 days post-inoculation/days post-contact to analyze transmission between directly inoculated and direct contact animals. Viral loads were analyzed by RT-qPCR. Virus titers are shown as the mean±SD Log₁₀ TCID₅₀ equivalent/mL. As seen in FIG. 8A and FIG. 8B viral RNA was detected in OP swabs and CL swabs collected from directly inoculated chickens from 1 to 6 dpi, with a trend towards lower viral RNA (vRNA) levels in chickens inoculated with MLV-H9N2-IL compared to those inoculated with MLV-H9N2. Notably, none of the direct contact chickens exhibited detectable levels of vRNA in either OP swabs or CL swabs at any time point. Blood samples were taken (n=4/group) at 14 dpi and 14 dpc and used for HI assay to analyze seroconversion. As seen in FIG. 9 serological analysis revealed no seroconversion in direct contact chickens at 14 days post-contact. These findings suggest that the replication levels of the MLVs in vivo are insufficient to allow for transmission to naïve direct contact birds.

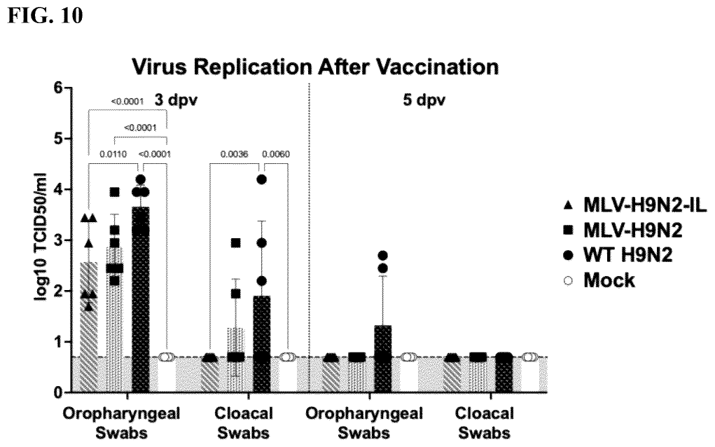

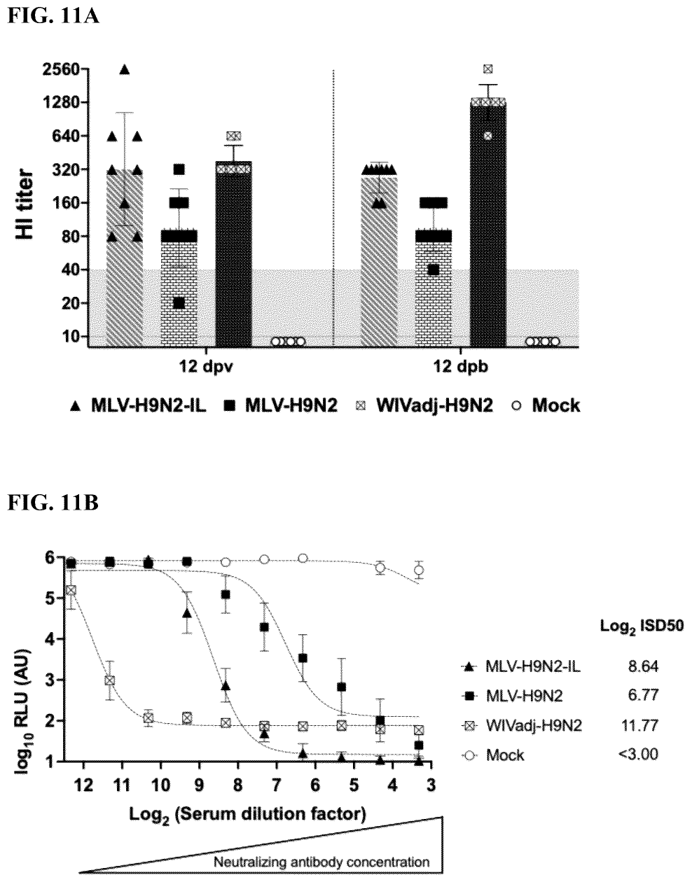

To evaluate the immunogenicity of MLV-H9N2 and MLV-H9N2-IL viruses, a prime-boost vaccination strategy administered two weeks apart was employed. Two-week-old chickens were inoculated via the EROC route with a dose of 1×10⁶ EID₅₀/chicken of either MLV-H9N2 or MLV-H9N2-IL. A control group received a subcutaneous (SC) injection of a WIVadj-H9N2 vaccine prepared in-house at a dose of 512 hemagglutinin units (HAU) per dose. A fourth group of chickens served as mock controls and were inoculated with PBS. OP swabs and CL swabs were collected after prime vaccination to assess virus replication levels. As seen in FIG. 10A, both MLV strains exhibited a limited window of replication, with detectable virus in both OP and CL swabs on day 3. Notably, MLV-H9N2-IL loads were significantly less than the H9N2 WT control in OP swabs on day 3, consistent with the reduced levels of viral RNA (vRNA) shown in FIG. 8A and FIG. 8B. Two weeks after boost, animals were challenged with 1×10⁸ EID₅₀/mL of the homologous virus via the EROC route. Samples were collected at the indicated time points to analyze virus replication after vaccination, serum antibody responses, and the protectiveness of MLVs after homologous challenge. No MLV virus was detected after boost vaccination.

To evaluate the protective efficacy of MLVs and the WIVadj-H9N2 vaccine, two weeks post-boost vaccination, chickens were challenged with a homologous virus at a dose of 1×10⁸ EID₅₀/chicken via the EROC route. CL and OP swabs were collected every other day until 7 dpc, and tissues were collected at 3 dpc. OP and CL swabs were taken (n=6/group) at 3-and 5-dpv to demonstrate the attenuated phenotype of the MLV-H9N2 and MLV-H9N2-IL compared to the H9N2 WT virus in vivo. In the graph in FIG. 10 data for MLV-H9N2 is shown in bars 2, 6, 10, and 14; data for MLV-H9N2-IL is shown in bars 1, 5, 9, and 13; data for H9N2 WT virus is shown in bars 3, 7, 11, and 15. Note that the MLV-H9N2-IL replicates significantly less than the H9N2 WT virus. Virus titers are shown as the mean±SD Log₁₀TCID₅₀/mL. Samples with undetected virus titers were assigned the limit of detection value (0.699 Log₁₀ TCID₅₀/mL). Blood samples were taken (n=8/group/timepoint) at 12 dpv and 12 dpb from the WIVadj-H9N2, MLV-H9N2, and MLV-H9N2-IL and used for hemagglutination inhibition (HI) assays, VNluc assays, and NP ELISA.

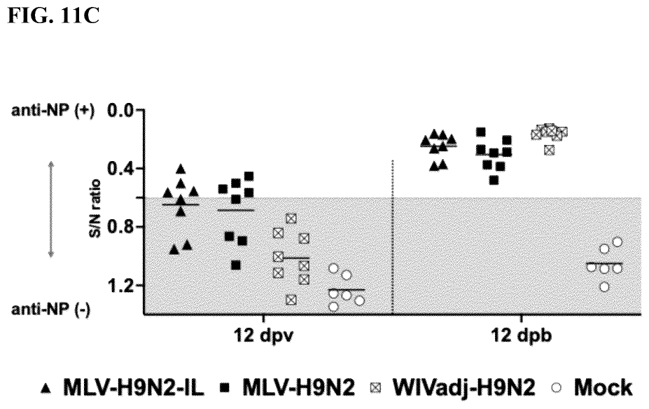

Serum samples collected at 12 dpv and 12 dpb revealed the presence of measurable HI and virus neutralization titers (VNluc), as well as anti-NP antibodies, in all vaccinated groups except for the PBS control group. The WIVadj-H9N2 vaccine group generated the highest HI titers after prime (mean titer of 320) and after boost (mean titer of 1280). Interestingly, as seen in FIG. 11A, the inclusion of IL-18 in the MLV-H9N2-IL vaccine resulted in higher HI titers after prime (mean titer of 320 versus 80) and after boost (mean titer of 320 versus 80) compared to the vaccine candidate without IL-18 (MLV-H9N2). To assess VNluc titers, a recombinant H9N1 homologous influenza virus expressing Nanoluciferase was employed, where titers are inversely proportional to its activity. In FIG. 11B, VNluc titers were plotted as the inhibitory sera dilution 50 (ISD₅₀) and were displayed in the graph. Consistent with the HI assay results, the WIVadj-H9N2 vaccine induced the highest levels of neutralizing antibodies (Log 10 ISD50 of 11.77), and MLV-H9N2-IL elicited higher levels of neutralizing antibodies (Log 10 ISD50 of 8.639) compared to MLV-H9N2 (Log 10 ISD₅₀ of 6.772), suggesting that IL-18 may play a role in enhancing the host immune response following vaccination and inducing higher levels of neutralizing antibodies indicative of protection. As seen in FIG. 11C, both MLVs induced higher levels of anti-NP antibodies after prime vaccination compared to the WIVadj-H9N2 vaccine, although all means were below the threshold for positive samples (0.6). After boost vaccination, anti-NP antibody levels increased in all groups and were comparable between the MLVs and the WIVadj-H9N2 vaccine. In conclusion, the findings disclosed herein demonstrate that MLVs are attenuated in vivo and induce neutralizing antibodies predictive of protection following vaccination.

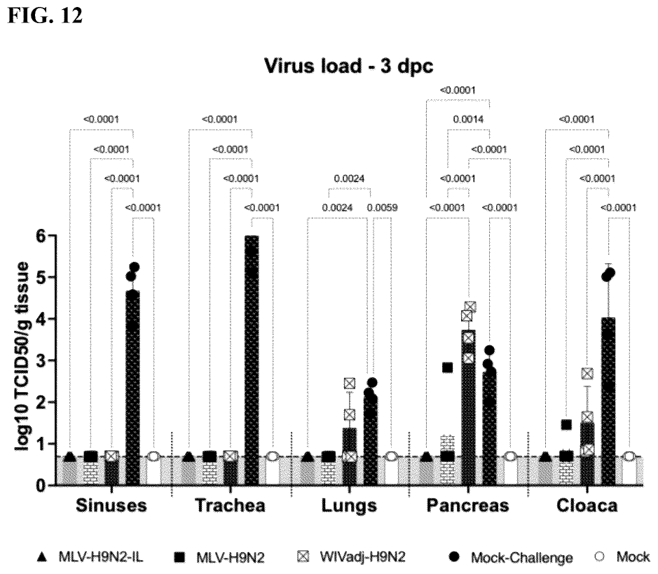

Sinuses, trachea, lungs, pancreas, and cloaca samples (n=4/group) were collected at 3 days post-challenge (dpc) and used to analyze the protection of vaccines by TCID₅₀. In FIG. 12 virus titers are shown as the mean±SD TCID50/g tissue. Data for MLV-H9N2-IL is shown in bars 1, 6, 11, 16, and 21; data for MLV-H9N2 is shown in bars 2, 7, 12, 17, and 22; data for WIVadj-H9N2 is shown in bars 3, 8, 13, 18, and 23; data for mock challenge is shown in bars 4, 9, 14, 19, and 24; and data for mock is shown in bars 5, 10, 15, 20, and 25. This figure shows that in the sinuses, trachea, and cloaca, all vaccines significantly reduced viral loads compared to the mock challenge. In the lungs, viral replication levels were similar between the WIVadj-H9N2 vaccine and mock challenge. Interestingly, viral replication levels in the pancreas were higher in the WIVadj-H9N2 vaccine group compared to the mock challenge, although the difference was not statistically significant. Both MLVs effectively reduced viral loads and completely protected the birds against the homologous virus in all tissues examined.

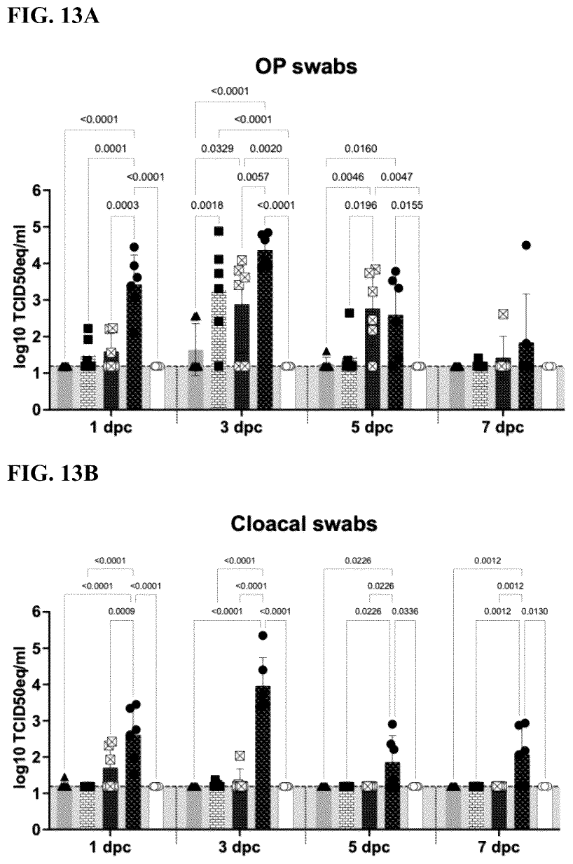

CL swabs (left) and OP swabs (right) were collected (n=6/group/day) every other day from 1 to 7 dpc to analyze the protection of vaccines by RT-qPCR. Virus titers are shown as the mean±SD Log 10 TCID50 equivalent/mL. Data for OP swabs is shown in FIG. 13A. In OP swabs, the MLV-H9N2-IL vaccine led to significant reduction in virus shedding. At 3 dpc the inclusion of IL-18 in the MLV-H9N2-IL vaccine significantly reduced viral loads and enhanced protection compared to the vaccine candidate without IL-18. Importantly, in OP swabs, the WIVadj-H9N2 vaccine did not reduce virus shedding at 3 and 5 dpc compared to the mock challenge. Data for CL swabs is shown in FIG. 13B. In CL swabs, all vaccines significantly reduced viral loads and fully protected the birds against a homologous challenge compared to the mock challenge. Overall, the findings shown here demonstrate that MLVs provide superior protection against a homologous challenge compared to the WIVadj-H9N2 vaccine. Data for MLV-H9N2-IL is shown in bars 1, 6, 11, and 16; data for MLV-H9N2 is shown in bars 2, 7, 12, and 17; data for WIVadj-H9N2 is shown in bars 3, 8, 13, and 18; data for mock challenge is shown in bars 4, 9, 14, and 19; and data for mock is shown in bars 5, 10, 15, and 20. Data analysis and graphs were prepared using Prism v9. Ordinary one-way ANOVA was performed to calculate P values followed by Tukey's multiple comparison test. Significant differences between vaccine groups are indicated by stars (*) as follows: * =p<0.05, ** =p<0.005, *** =p<0.0005, and **** =p<0.0001. Some figures were created with BioRender.com.

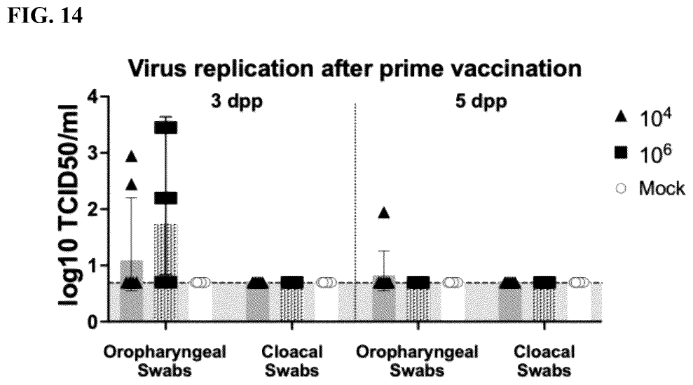

To assess the feasibility of mass vaccination using the most promising vaccine candidate, MLV-H9N2-IL, two-week-old chickens were vaccinated via drinking water for 2 hours with either 1×10⁴ EID₅₀/mL (low dose) or 1×10⁶ EID₅₀/mL (high dose) of MLV-H9N2-IL, employing either a prime-only or prime-boost strategy. OP and CL swabs were collected to evaluate virus replication levels following drinking water vaccination. As seen in FIG. 14, MLV-H9N2-IL exhibited dose-dependent and limited virus replication in OP swabs on day 3 post-vaccination. Infectious virus was not detected in CL swabs.

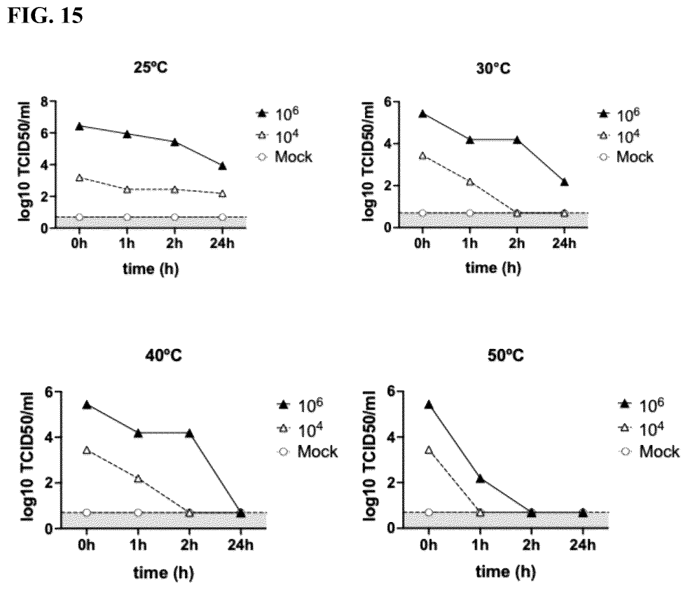

To assess vaccine stability in drinking water, vaccine samples were mixed with distilled water and incubated at various temperatures (25° C., 30° C., 40° C., and 50° C.) for 2 hours. Chickens were allowed to drink water for 2 hours, and as seen in FIG. 15, during this period, MLV-H9N2-IL was found to be stable at both low and high dose at 25° C. Additionally, at 30° C. and 40° C., the MLV-H9N2-IL high dose remained stable for 2 hours, while low dose appeared to decrease, with no infectious virus detected after 2 hours at these temperatures. Finally, titers rapidly declined when samples were incubated at 50° C.

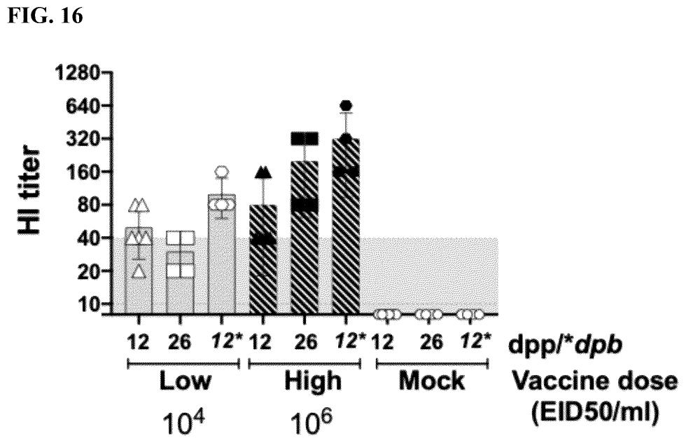

Blood samples were collected at 12 dpv and 12 dpb to measure HI titers, neutralizing antibodies, and anti-NP antibodies. For the prime-only groups, blood was also collected at 26 dpv, two days prior to the challenge. As can be seen in FIG. 16, in the low dose group, HI titers increased from around 40 (20-80) after prime to 80 (80-160) following the boost. Similarly, HI titers increased in the high dose group after the boost (average 320, 160-640) compared to prime (average 80, 40-160). Notably, HI titers increased from 80 to 160 when comparing 12 dpv to 26 dpv in the high dose group.

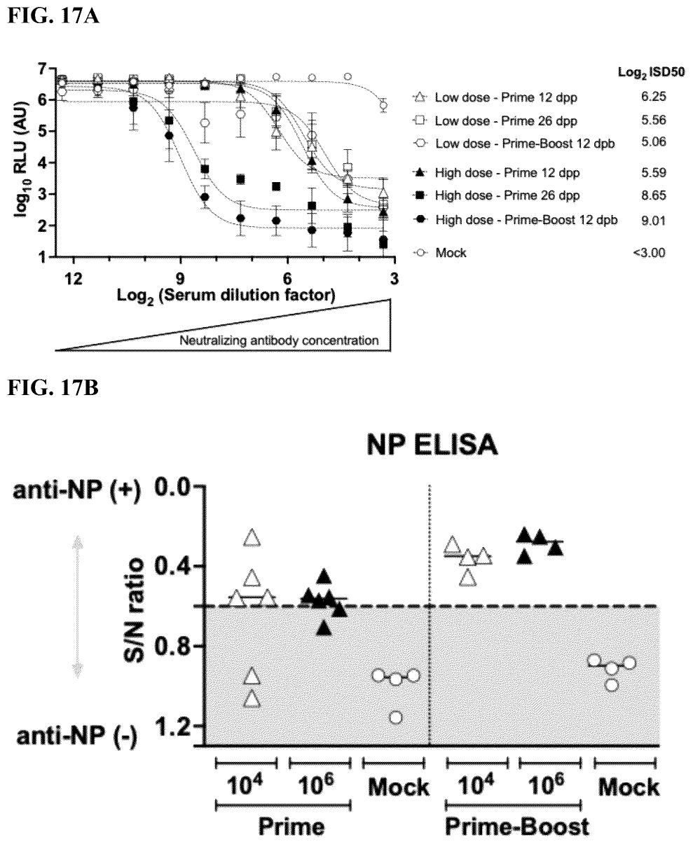

Virus neutralization data is shown in FIG. 17A. Consistent with the HI data, VNluc assays revealed similar and consistent results, with a prime-only (Log 10 ISD₅₀ of 8.649) or prime-boost (Log 10 ISD₅₀ of 9.014) regimen using high dose generating the highest levels of neutralizing antibodies. As seen in FIG. 17B, anti-NP antibodies were also detected after prime vaccination and increased after boost, with comparable levels observed between the two doses tested. In summary, these findings demonstrate that MLV-H9N2-IL is stable during vaccine administration, remains attenuated when administered via drinking water, and induces neutralizing antibodies predictive of protection following vaccination.

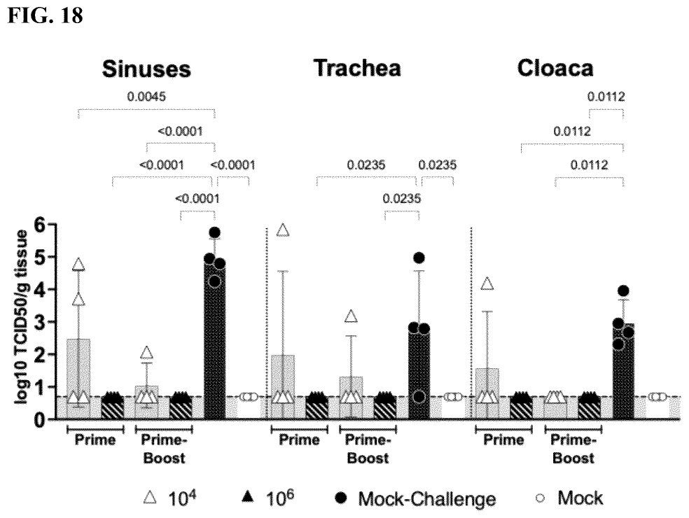

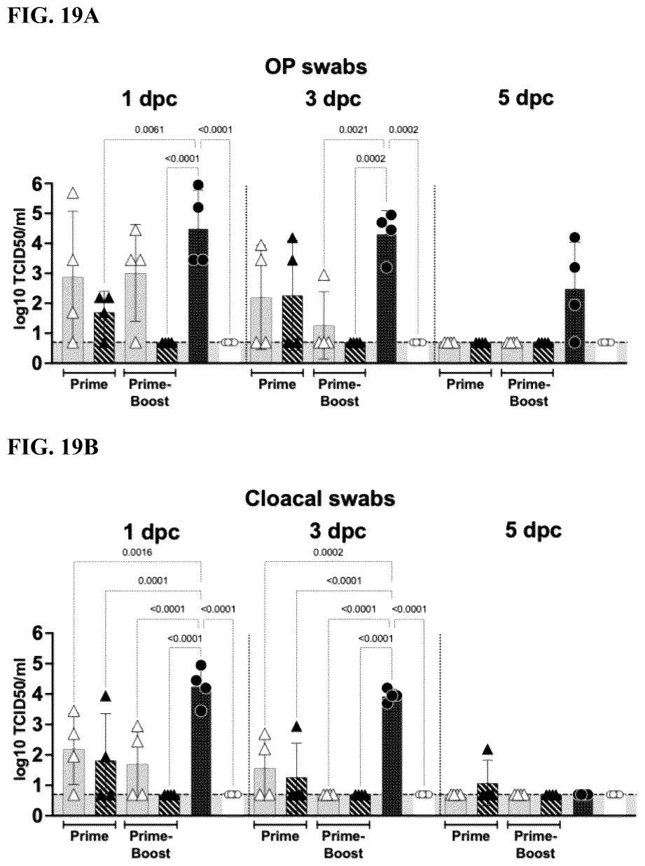

Two weeks after the boost vaccination or four weeks after the prime vaccination, chickens were challenged with a homologous virus at a dose of 1×10⁸ EID₅₀/chicken via the EROC route. OP and CL swabs were collected every other day until 5 dpc, and tissues were collected at 3 dpc. As seen in FIG. 18, tissue analysis revealed that all doses and vaccination strategies protected against the homologous challenge by reducing viral loads in the sinuses. Similar results were observed in the cloaca. In the trachea, a decrease in viral loads was observed compared to the mock challenge, but the difference was not statistically significant. As seen in FIG. 19A, in OP swabs, prime-only and prime-boost vaccination with high dose significantly reduced viral loads compared to the mock challenge at 1 dpc. Prime-only or prime-boost vaccination with low dose also decreased viral loads but lacked statistical significance. On 3 dpc, a boost vaccination was required to significantly reduce viral loads compared to the mock challenge, regardless of the dose. All doses and strategies significantly reduced viral loads at 5 dpc. Importantly, prime-boost vaccination with high dose completely prevented virus shedding in OP swabs on days 1, 3, and 5 dpc.

As shown in FIG. 19B, in CL swabs, all vaccine doses, and strategies (low dose prime-only, high dose prime-only, low dose prime-boost, and high dose prime-boost) significantly reduced viral loads and protected the birds against the homologous virus at 1 and 3 dpc. Notably, vaccination with a prime-boost regimen at high dose completely prevented virus shedding in CL swabs after the challenge. In FIG. 19A and FIG. 19B bars 1, 3, 7, 9, 13 and 15 show data for 10⁴ EID₅₀/mL; bars 2, 4, 8, 10, 14, and 16 show data for 10⁶ EID₅₀/mL; bars 5, 11, and 17 show data for mock-challenge; and bars 6, 12, and 18 show data for Mock.

In conclusion, prime-boost vaccination with high dose of MLV-H9N2-IL via drinking water induces sterilizing immunity in chickens, as evidenced by the absence of detectable virus replication in OP swabs, CL swabs, and the sinuses, trachea, and cloaca.

We claim:

1. A recombinant influenza virus comprising:
a modified gene segment 2 with at least one of a polynucleotide encoding a histidine tag or a polynucleotide encoding M42 at the C-terminus of the PB1 open reading frame (ORF); or
a modified gene segment 4 containing an IgA-inducing protein (IGIP) coding sequence;
or
a modified gene segment 6 containing a chicken IL-18 coding sequence.

2. The recombinant influenza virus of claim 1, wherein the modified gene segment 2 further comprises at least one of K391E, D581G, and A661T temperature sensitive mutations.

3. The recombinant influenza virus of claim 1, wherein the modified gene segment 2 has a polynucleotide encoding a histidine tag at the C-terminus of the PB1 ORF.

4. The recombinant influenza virus of claim 1, further comprising a gene segment 1 comprising at least one temperature sensitive mutation.

5. The recombinant influenza virus of claim 1, further comprising a mutated gene segment 7, wherein the mutated gene segment 7 does not express M2.

6. The recombinant influenza virus of claim 1, wherein the recombinant influenza virus comprises a modified gene segment 2 with a K391E, a D581G, and an A661T temperature sensitive mutations, a polynucleotide encoding a histidine tag at the C-terminus of the PB1 ORF; a gene segment 1 with at least one temperature sensitive mutation; and a mutated gene segment 7 which does not express M2.

7. The recombinant influenza virus of claim 1, wherein the modified gene segment 2 has a polynucleotide encoding M42 at the C-terminus of the PB1 ORE.

8. The recombinant influenza virus of claim 7, further comprising a modified gene segment 7 which does not express M2 or M42.

9. A cell comprising a recombinant influenza virus of claim 1.

10. The cell of claim 9, wherein the cell is selected from the group consisting of MDCK cells, Vero cells, CV-1 cells, LLcomk.2 cells, MDBK cells, BK-1 cells, Chinese Hamster Ovary cells, 293T cells, human embryonic kidney cells, avian embryonic fibroblasts, and in ovo.

11. A composition comprising a recombinant influenza virus of claim 1, and optionally an adjuvant.

12. A method of treating a vertebrate in need thereof to prevent or ameliorate one or more symptoms of influenza virus infection, the method comprising administering to the subject a recombinant influenza virus comprising:
a modified gene segment 2 with at least one of a polynucleotide encoding a histidine tag or a polynucleotide encoding M42 at the C-terminus of the PB1 open reading frame (ORF); or
a modified gene segment 4 containing an IgA-inducing protein (IGIP) coding sequence;
or
a modified gene segment 6 containing a chicken IL-18 coding sequence; and
optionally a carrier.

13. The method of claim 12, wherein the vertebrate are birds, Canidae, Cetacea, Felidae, Mustelidae, Rodentia, Equidae, Bovidae, Suidae, or Primates.

14. The method of claim 13, wherein the birds are water fowl, chickens, or turkeys.

15. The method of claim 12, wherein the vertebrates are mammals.

16. The method of claim 15, wherein the mammals are pigs, horses, whales, dolphins, or humans.


SEQUENCE LISTING (14 sequences)

SEQ ID NO: 1 - protein, 5 aa, synthetic construct
GGGGS

SEQ ID NO: 2 - protein, 8 aa, synthetic construct
DRPAVIAN

SEQ ID NO: 3 - DNA, 32 bp, synthetic construct
  source 1..32: mol_type = other DNA
ttcttgaggatgaacagatgtaccagaagtgc

SEQ ID NO: 4 - DNA, 40 bp, synthetic construct
  source 1..40: mol_type = other DNA
atatcgtctcgtattagtagaaacaaggcattttttcatg

SEQ ID NO: 5 - DNA, 23 bp, synthetic construct
  source 1..23: mol_type = other DNA
ctagcagttaaccggagtactgg

SEQ ID NO: 6 - DNA, 37 bp, synthetic construct
  source 1..37: mol_type = other DNA
gacaaggggccttggccctatcactggtttgaaggtc

SEQ ID NO: 7 - DNA, 20 bp, synthetic construct
  source 1..20: mol_type = other DNA
gattcgcgctctggttatga

SEQ ID NO: 8 - DNA, 22 bp, synthetic construct
  source 1..22: mol_type = other DNA
agtagaaacaaggagttttttc

SEQ ID NO: 9 - DNA, 24 bp, synthetic construct
  source 1..24: mol_type = other DNA
agatgagtcttctaaccgaggtcg

SEQ ID NO: 10 - DNA, 24 bp, synthetic construct
  source 1..24: mol_type = other DNA
tgcaaaaacatcttcaagtctctg

SEQ ID NO: 11 - DNA, 17 bp, synthetic construct
  source 1..17: mol_type = other DNA
ggccccctcaaagccga

SEQ ID NO: 12 - DNA, 22 bp, synthetic construct
  source 1..22: mol_type = other DNA
gttacgcgccagcaaaagcagg

SEQ ID NO: 13 - DNA, 22 bp, synthetic construct
  source 1..22: mol_type = other DNA
gttacgcgccagcgaaagcagg

SEQ ID NO: 14 - DNA, 23 bp, synthetic construct
  source 1..23: mol_type = other DNA
gttacgcgccagtagaaacaagg